(19) United States
(12) Patent Application Publication
(10) Pub. No.: US 2011/0053198 A1
(43) Pub. Date: Mar. 3, 2011

(54) UBIQUITIN PROTEASOME SYSTEM PROFILING AND THE USE THEREOF IN CLINICAL APPLICATIONS FOR PROLIFERATIVE HEMATOLOGICAL DISORDERS

(75) Inventors: Maher Albitar, Coto De Caza, CA (US); Wanlong Ma, Aliso Viejo, CA (US); Ke Zhang, Thousand Oaks, CA (US)

(73) Assignee: Quest Diagnostics Investments Incorporated

(21) Appl. No.: 12/547,455

(22) Filed: Aug. 25, 2009

(51) Int. Cl. C12Q 1/37; C12Q 1/02

(52) U.S. Cl. 435/24; 435/29

(57) ABSTRACT

Provided herein are methods for the diagnosis, prognosis, or management of proliferative hematological disorders and other diseases using profiles of the ubiquitin-proteasome system determined from acellular body fluids or cell-containing samples. Further provided are methods of predicting response to therapy in certain populations of leukemia patients.

### FIELD OF THE INVENTION

The invention relates to the diagnosis, prognosis, and management of hematological disorders, including leukemia.

### BACKGROUND OF THE INVENTION

The following discussion of the background of the invention is merely provided to aid the reader in understanding the invention and is not admitted to describe or constitute prior art to the present invention.

The ubiquitin-proteasome system (UPS) is responsible for the degradation of approximately 80-90% of normal and abnormal intracellular proteins and therefore plays a central role in a large number of physiological processes. For example, the regulated proteolysis of cell cycle proteins, including cyclins, cyclin-dependent kinase inhibitors, and tumor suppressor proteins, is required for controlled cell cycle progression and proteolysis of these proteins occurs via the ubiquitin-proteasome pathway (Deshaies, Trends in Cell Biol., 5:428-434 (1995) and Hoyt, Cell, 91:149-151 (1997)). In another example, the activation of the transcription factor NF-κB, which itself plays a central role in the regulation of genes involved in the immune and inflammatory responses, is dependent upon the proteasome-mediated degradation of an inhibitory protein, IκαB-α (Palombella et al., WO 95/25533). In yet another example, the ubiquitin-proteasome pathway plays an essential role in antigen presentation through the continual turnover of cellular proteins (Goldberg and Rock, WO 94/17816).

While serving a central role in normal cellular homeostasis, the UPS also mediates the inappropriate or accelerated protein degradation occurring as a result or cause of pathological conditions including cancer, inflammatory diseases, and autoimmune diseases, characterized by deregulation of normal cellular processes. In addition, the cachexia or muscle wasting associated with conditions such as cancer, chronic infectious diseases, fever, muscle atrophy, nerve injury, renal failure, and hepatic failure results from an increase in proteolytic degradation by the UPS (Goldberg, U.S. Pat. No. 5,340,736 (1994)). Furthermore, the cytoskeletal reorganization that occurs during maturation of protozoan parasites is proteasome-dependent (Gonzales et al., J. Exp. Med., 184:1909 (1996)).

Central to this system is the 26S proteasome, a multi-subunit proteolytic complex, consisting of one 20S proteasome core and two flanking 19S complexes. The 20S proteasome consists of four rings: two outer α-rings and two inner β-rings surrounding a barrel-shaped cavity. The two inner β-rings form a central chamber that harbors the catalytic site for the chymotryptic, tryptic, and caspase-like activities (von Mikecz, J Cell Sci, 119(10):1977-84, 2006).

Proteins targeted for degradation by the proteasome contain a recognition signal. This signal consists of a polyubiquitin chain that is selectively attached to the protein target by the sequential addition of ubiquitin monomers. The polyubiquitin signal is recognized by the 19S complex, which mediates the entry of the target protein into the protcolytic chamber.

### SUMMARY OF THE INVENTION

The present invention is based on the discovery that the specific activity of proteasomal peptidases may be detected in patient samples and that such activity can have clinical value in the diagnosis and prognosis of certain disease states.

In one aspect, the invention provides a method for diagnosing a proliferative hematological disorder in a subject, the method comprising: determining, in a body fluid sample (e.g., an acellular body fluid sample) from the subject, the specific activity of one or more (i.e. one, two, or three) proteasomal peptidases selected from the group consisting of chymotrypsin-like activity (Ch-L), trypsin-like activity (Tr-L), and caspase-like activity (Cas-L), wherein the specific activity is determined by normalizing the one or more peptidase activities to the amount of proteasomal protein in the sample, and wherein a difference of the specific activity of one or more proteasomal peptidases compared to a reference level indicates a proliferative hematological disorder in the subject. In one embodiment, the acellular body fluid is selected from the group consisting of serum and plasma.

In certain embodiments, an increase or decrease in the specific activity of one or more proteasomal peptidases relative to the corresponding specific activity in a comparable sample from one or more healthy individuals is a factor favoring diagnosis of a proliferative hematological disorder, e.g., acute myeloid leukemia (AML) myelodysplastic syndrome (MDS), or acute lymphoblastic leukemia (ALL).

In suitable embodiments, the determined specific activity can he compared to a reference value. In some embodiments, the reference value for each specific activity can be the specific activity for each peptidase in a comparable sample from one or more healthy individuals. In a particular embodiment, the reference value is a cutoff value that has been statistically calculated based on specific activities determined from a particular population of individuals (e.g., a population of AML patients) or based on a statistical model to determine a cutoff value for predicting a specific clinical behavior. In this embodiment, a determined specific activity greater than or lower than a cutoff value is related to an unfavorable diagnosis for the patient. In some embodiments, a determined specific activity in the patient sample that is the same as or substantially the same as the specific activity in the reference sample (i.e., a comparable acellular body fluid sample from one or more healthy individuals) reflects a positive prognosis for the patient.

In one embodiment, a determined specific activity of Ch-L (Ch-L/p) in the subject sample that is lower than a reference value indicates a diagnosis of AML or ALL for the subject. In one embodiment, a determined level of specific activity of Cas-L (Cas-L/p) in the subject sample that is higher than a reference value indicates a diagnosis of MDS for the subject. In one embodiment, a determined level of specific activity of Cas-L (Cas-L/p) in the subject sample that is lower than a reference value indicates a diagnosis of ALL for the subject. In one embodiment, a determined level of specific activity of Tr-L (Tr-L/p) in the subject sample that is lower than a reference value indicates a diagnosis of ALL for the subject. In one embodiment, a determined level of specific activity of Tr-L (Tr-L/p) in the subject sample that is higher than a reference value indicates a diagnosis of MDS for the subject.

In one aspect, the present invention provides a method of diagnosing a proliferative hematological disorder in a subject, the method comprising: determining the amount of one or more proteasomal proteins in a test sample for the subject; determining the amount of one or more (i.e., one, two, or three) proteasomal peptidase activities in a test sample from the subject, the peptidase activities selected from the group consisting of chymotrypsin-like activity (Ch-L), trypsin-like activity (Tr-L), and caspase-like activity (Cas-L), normalizing the amount of one or more proteasomal peptidase activities to the amount of proteasomal protein to provide a specific activity of the one or more proteasomal peptidases; and using the specific activity of the one or more proteasomal peptidases to diagnose the presence of a proliferative hematological disorder in the subject.

In another aspect, the invention provides a method of determining a prognosis of a subject having a proliferative hematological disorder, wherein the method comprises: determining the specific activity of one or more (i.e., one, two, or three) proteasomal peptidases selected from the group consisting of chymotrypsin-like activity (Ch-L), trypsin-like activity (Tr-L), and caspase-like activity (Cas-L), wherein the specific activity is determined by normalizing the one or more proteasomal peptidases activities to a amount of proteasomal protein in the sample, and wherein a difference of the specific activity of one or more peptidase activities is compared to a reference level indicates the prognosis of a subject suffering from a proliferative hematological disorder. In one embodiment, the prognosis is selected from the group consisting of survival rate, 5-year survival rate, and complete remission duration (CRD).

In one embodiment, the reference level is the level of specific activity of corresponding proteasomal proteins in a comparable sample from one or more healthy individuals. In one embodiment, the test sample is a cell-containing sample. In another embodiment, the test sample is an acellular body fluid sample, e.g., serum or plasma. In one embodiment, the specific activity of Ch-L (Ch-L/p) in the subject sample that is higher than a reference value reflects a better survival rate from ALL for the subject.

In another aspect, the invention provides a method of determining a prognosis of a subject having a proliferative hematological disorder, wherein the method comprises determining the level of circulating ubiquitin or polyubiquitin in a sample from the subject, and providing a prognosis for the subject based on a difference of the level of circulating ubiquitin or polyubiquitin compared to a reference level. In one embodiment, the proliferative hematological disorder is CLL.

In one embodiment, a level of circulating ubiquitin or polyubiquitin greater than about 192 ng/mL indicates a better survival rate for the subject compared to subjects having a level of circulating ubiquitin or polyubiquitin less than about 192 ng/mL.

In one embodiment, the methods further comprise determining the level of beta-2 microglobulin in a sample from the subject. In one embodiment, a level of beta-2 microglobulin less than about 3.2 mg/L indicates a better survival rate for the subject compared to subjects having a level of circulating beta-2 microglobulin greater than about 3.2 mg/L.

### BRIEF DESCRIPTION OF THE FIGURES

FIG. 1 is a series of charts showing high levels of proteasome, ubiquitin, and proteasome activity in patients with leukemia and myelodysplastic syndrome. Box plots showing levels of proteasome (FIG. 1A), ubiquitin (FIG. 1B), Ch-L activity (FIG. 1C), cas-L activity (FIG. 1D), and Tr-L activity (FIG. 1E) in acute lymphoblastic leukemia (ALL), acute myeloid leukemia (AML), myelodysplastic syndrome (MDS), and healthy controls (n). The P-values between adjacent groups is shown. Asterisks and circles indicate outliers and extreme values, respectively.

FIG. 2 is a series of charts showing relatively low enzymatic activity of proteasomes in acute leukemias despite high number of proteasomes. Normalized Ch-L (Ch-L/p), normalized Cas-L (Cas-L/p), and normalized Tr-L (Tr-L/p) are shown in FIG. 2A, FIG. 2B, and FIG. 2C, respectively. Abbreviations: ALL, acute lymphoblastic leukemia; AML, acute myeloid leukemia; MDS, myelodysplastic syndrome; and n, healthy control. The P-values between adjacent groups is shown. Asterisks and circles indicate outliers and extreme values, respectively.

FIG. 3 is a chart showing Kaplan-Meier estimates of patient survival grouped by plasma proteasome protein levels in all patients with acute myeloid leukemia. Patients with proteasome levels higher than the median 875 ng/mL show significantly shorter survival (P=0.04) N:E indicates the total number of patients and the number of patients with an event (death).

FIG. 4 is a chart showing Kaplan-Meier estimates of patient survival grouped by plasma proteasome protein levels in patients with acute myeloid leukemia and unfavorable cytogenetic abnormalities. Patients with proteasome levels higher than 875 ng/ml show significantly shorter survival. N:E indicates the total number of patients and the number of patients with an event (death).

FIG. 5 is a chart showing Kaplan-Meier estimates of patient survival grouped by plasma normalized Ch-L activity (Ch-L/p) levels in patients with acute lymphoblastic leukemia. Patients with Ch-L/p level higher than the median 0.88 pMol AMC/Sec/pg proteasome show significantly better survival.

FIG. 6 is a chart showing Kaplan-Meier estimates of patient survival grouped by beta-2 microglobulin (B2M) and poly-ubiquitin levels in patients with chronic lymphocytic leukemia. Patients with ubiquitin levels higher than 192 ng/ml have significantly better survival

### DETAILED DESCRIPTION

The present invention relates generally to methods of assessing the ubiquitin-proteasome system (UPS) for the diagnosis of disease. As demonstrated herein, increasing or decreasing amounts of the specific activity of one or more proteasomal peptidases correlates with the presence of disease or the prognosis of a patient suffering from a disease. In particular, methods for diagnosing proliferative hematological disorders, determining the likelihood of survival, and methods for predicting likelihood for responsiveness to therapy are provided.

The present technology is described herein using several definitions, as set forth throughout the specification. As used herein, unless otherwise stated, the singular forms “a,” “an,” and “the” include plural reference. Thus, for example, a reference to “a proteasome” is a reference to one or more proteasomes.

The term “about” as used herein in reference to quantitative measurements or values, refers to the enumerated value plus or minus 10%, unless otherwise indicated.

The term “antibody” as used herein encompasses both monoclonal and polyclonal antibodies that fall within any antibody classes, IgG, IgM, IgA, IgE, or derivatives thereof. The term “antibody” also includes antibody fragments including, but not limited to, Fab, F(ab′)₂, and conjugates of such fragments, and single-chain antibodies comprising an antigen recognition epitope. In addition, the term “antibody” also means humanized antibodies, including partially or fully humanized antibodies. An antibody may be obtained from an animal, or from a hybridoma cell line producing a monoclonal antibody, or obtained from cells or libraries recombinantly expressing a gene encoding a particular antibody.

The terms “assessing” and “evaluating” are used interchangeably to refer to any form of measurement, and include determining if a characteristic, trait, or feature is present or not. The terms “determining,” “measuring,” “assessing,” and “assaying” are used interchangeably and include both quantitative and qualitative determinations. Assessing may be relative or absolute. “Assessing the presence of” includes determining the amount of something present, as well as determining whether it is present or absent.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm and sputum. A body fluid sample may be collected by any suitable method. The body fluid sample may be used immediately or may be stored for later use. Any suitable storage method known in the art may be used to store the body fluid sample; for example, the sample may be frozen at about −20° C. to about −70° C. Suitable body fluids arc acellular fluids. “Acellular” fluids include body fluid samples in which cells are absent or are present in such low amounts that the peptidase activity level determined reflects its level in the liquid portion of the sample, rather than in the cellular portion. Typically, an acellular body fluid contains no intact cells. Examples of acellular fluids include plasma or serum, or body fluids from which cells have been removed.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes (e.g., liver enzymes), examination of blood cells or bone marrow cells, cytogenetics, and immunophenotyping of blood cells. Specific activity of one or more proteasomal peptidases is a clinical factor.

The term “comparable” or “corresponding” in the context of comparing two or more samples, means that the same type of sample (e.g., plasma) is used in the comparison. For example, a specific activity level of one or more proteasomal peptidases in a sample of plasma can he compared to a specific activity level in another plasma sample. In some embodiments, comparable samples may be obtained from the same individual at different times. In other embodiments, comparable samples may be obtained from different individuals (e.g., a patient and a healthy individual). In general, comparable samples are normalized by a common factor. For example, acellular body fluid samples are typically normalized by volume body fluid and cell-containing samples are normalized by protein content or cell count.

As used herein, the term “diagnosis” means detecting a disease or disorder or determining the stage or degree of a disease or disorder. Usually, a diagnosis of a disease or disorder is based on the evaluation of one or more factors and/or symptoms that arc indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The term “diagnosis” also encompasses determining the therapeutic effect of a drug therapy, or predicting the pattern of response to a drug therapy. The diagnostic methods may be used independently, or in combination with other diagnosing and/or staging methods known in the medical art for a particular disease or disorder, e.g., a proliferative hematological disorder.

As used herein, the phrase “difference of the level” refers to differences in the quantity of a particular markers, such as a protein or protein activity, in a sample as compared to a control or reference level. For example, the quantity of particular protein and/or the amount of a protein activity may he present at an elevated amount or at a decreased amount in samples of patients with a proliferative hematological disorder compared to a reference level. In one embodiment, a “difference of a level” may be a difference between the specific activity of a proteasomal peptidase present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In one embodiment, a “difference of a level” may be a statistically significant difference between the specific activity of a proteasomal peptidase present in a sample as compared to a control. For example, a difference may be statistically significant if the measured level of the specific activity falls outside of about 1.0 standard deviations, about 1.5 standard deviations, about 2.0 standard deviations, or about 2.5 stand deviations of the mean of any control or reference group.

The term “enzyme linked immunosorbent assay” (ELISA) as used herein refers to an antibody-based assay in which detection of the antigen of interest is accomplished via an enzymatic reaction producing a detectable signal. ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generated a detectable product upon digestion of appropriate substrates.

The term “label” as used herein, refers to any physical molecule directly or indirectly associated with a specific binding agent or antigen which provides a means for detection for that antibody or antigen. A “detectable label” as used herein refers any moiety used to achieve signal to measure the amount of complex formation between a target and a binding agent. These labels are detectable by spectroscopic, photochemical, biochemical, immunochemical, electromagnetic, radiochemical, or chemical means, such as fluorescence, chemifluoresence, or chemiluminescence, electrochemiluminescence or any other appropriate means. Suitable detectable labels include fluorescent dye molecules or fluorophores.

The term “proliferative hematological disorder” as used herein means a disorder of a bone marrow or lymph node-derived cell type, such as a white blood cell. A proliferative hematological disorder is generally manifest by abnormal cell division resulting in an abnormal level of a particular hematological cell population. The abnormal cell division underlying a proliferative hematological disorder is typically inherent in the cells and not a normal physiological response to infection or inflammation. A leukemia is a type of proliferative hematological disorder. Exemplary proliferative hematological disorders include, but are not limited to, acute myeloid leukemia, acute lymphoblastic leukemia, chronic lymphocytic leukemia, myelodysplastic syndrome, chronic myeloid leukemia, hairy cell leukemia, leukemic manifestations of lymphomas, and multiple myeloma. Lymphoma is a type of proliferative disease that mainly involves lymphoid organs, such as lymph nodes, liver, and spleen. Exemplary proliferative lymphoid disorders include lymphocytic lymphoma (also called chronic lymphocytic leukemia), follicular lymphoma, large cell lymphoma, Burkitt's lymphoma, marginal zone lymphoma, lymphoblastic lymphoma (also called acute lymphoblastic lymphoma).

The term “prognosis” as used herein refers to a prediction of the probable course and outcome of a clinical condition or disease. A prognosis is usually made by evaluating factors or symptoms of a disease that are indicative of a favorable or unfavorable course or outcome of the disease. The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better than average cure rate, a lower propensity for metastasis, a longer than expected life expectancy, differentiation of a benign process from a cancerous process, and the like. For example, a positive prognosis is one where a patient has a 50% probability of being cured of a particular cancer after treatment, while the average patient with the same cancer has only a 25% probability of being cured.

As used herein, “plasma” refers to acellular fluid found in blood. Plasma may be obtained from blood by removing whole cellular material from blood by methods known in the art (e.g., centrifugation, filtration, and the like). As used herein, “peripheral blood plasma” refers to plasma obtained from peripheral blood samples.

As used herein, “serum” includes the fraction of plasma obtained after plasma or blood is permitted to clot and the clotted fraction is removed.

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide.

As used herein, the term “proteasome” refers to certain large protein complexes within cells or body fluid that degrade proteins that have been tagged for elimination, particularly those tagged by ubiquitination. Proteasomes degrade denatured, misfolded, damaged, or improperly translated proteins. Proteasomes degradation of certain proteins, such as cyclins and transcription factors, serves to regulate the levels of such proteins. Exemplary proteasomes include the 26S proteasome, 20S proteasome, and the immunoproteasome.

The “26S proteasome” consists of 3 subcomplexes. The 26S proteasome consists of a 20S proteasome at the core which is capped at each end by a 19S regulatory particle (RP or PA700). The 19S RP mediates the recognition of the ubiquitinated target proteins, the ATP-dependent unfolding and the opening of the channel in the 20S proteasome, allowing entry of the target protein into the proteolytic chamber.

The “20S proteasome,” which forms the core protease (CP) of the 26S proteasome, is a barrel-shaped complex consisting of four stacked rings, each ring having 7 distinct subunits. The four rings are stacked one on top of the other and are responsible for the proteolytic activity of the proteasome. There are two identical outer α rings, having no known function, and two inner β rings, containing multiple catalytic sites. In eukaryotes, two of these sites on the β rings have chymotrypsin-like activity (Ch-L), two of these sites have trypsin-like activity (Tr-L), and two have caspase-like activity (Cas-L).

The “immunoproteasome,” which is characterized by an ability to generate major histocompatibility complex class I-binding peptides, consists of a 20S proteasome core capped on one end by 19S RP and on the other end by PA28, an activator of the 20S proteasome and an alternative RP. PA28 consists of two homologous subunits (termed α and β) and a separate but related protein termed PA28γ (also known as the Ki antigen).

The term “proteasomal peptidase activity” refers to any proteolytic enzymatic activity associated with a proteasome, such as the 26S or 20S proteasomes. The peptidase activities of proteasomes include, for example, chymotrypsin-like activity (Ch-L), trypsin-like activity (Tr-L), and caspase-like activity (Cas-L). In some embodiments, proteasomal peptidase activity is determined by measuring the rate of cleavage of a substrate per unit volume of body fluid assayed. Thus, the activity may be expressed as (moles of product formed)/time/(volume body fluid). For example, the activity may be expressed as pmol/sec/mL.

As used herein, the term “reference level” refers to a level of a substance which may be of interest for comparative purposes. In one embodiment, a reference level may be the specific activity level of a proteasomal peptidase expressed as an average of the level of the specific activity of the proteasomal peptidase from samples taken from a control population of healthy (disease-free) subjects. In another embodiment, the reference level may be the level in the same subject at a different time. e.g., before the present assay such as the level determined prior to the subject developing the disease or prior to initiating therapy. In general, samples arc normalized by a common factor. For example, acellular body fluid samples are normalized by volume body fluid and cell-containing samples are normalized by protein content or cell count.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascites fluid, or whole blood, and including biopsy samples of body tissue. A sample may be obtained from any subject, e.g., a subject/patient having or suspected to have a proliferative hematological disorder.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with proliferative hematological disorder.

As used herein, the term “specific activity” of one or more proteasomal peptidases refers to the proteasomal peptidase activity in the sample that is normalized relative to the proteasomal protein content in the sample. Specific activity of the chymotrypsin-like, trypsin-like, and caspase-like proteasomal peptidases may be designated Ch-L/p, Tr-L/p, or Cas-L/p, respectively. The skilled artisan understands that normalization of the proteasomal peptidase activity to the proteasomal protein content in the sample involves measuring and expressing the amount of proteasomal protein per unit volume of body fluid assayed, in the same type of sample (preferably a split sample) as used to measure enzymatic activity. For example, proteasomal protein may he expressed as picograms (pg) of protein per mL which, when used to normalize a proteasomal peptidase activity expressed in pmol/sec/mL, results in a specific activity expressed in pmol/sec/pg proteasomal protein.

The phrase “substantially the same as” in reference to a comparison of one value to another value for the purposes of clinical management of a disease or disorder means that the values are statistically not different. Differences between the values can vary, for example, one value may be within 20%, within 10%, or within 5% of the other value.

As used herein, the term “UPS Score” refers to a single number or score, based on a statistical analysis of the measured level of one or more biomarkers selected from the group consisting of Ch-L/p, Cas-L/p, and Tr-L/p, that reflects a relationship of a specific subject to any one particular group of individuals, such as normal individuals or individuals having a disease or any progressive state thereof. In some embodiments, the UPS score is derived from a quantitative multivariate analysis, which reflects the overall statistical assessment of an individual patient's clinical condition based upon an integrated statistical calculation of a plurality of qualitatively unique factors, e.g., specific activity of proteasomal peptidases, proteasome level, age, gender, etc.

### Overview

Disclosed herein are methods for detecting the presence or absence of proliferative hematological disorders in subjects based, at least in part, on results of testing methods of the present technology on a sample. Further disclosed herein are methods for monitoring the status of subjects diagnosed with proliferative hematological disorders based at least partially on results of tests on a sample. The test samples disclosed herein are represented by, but not limited in anyway to, sputum, blood (or a fraction of blood such as plasma, serum, or particular cell fractions), lymph, mucus, tears, saliva, urine, semen, ascites fluid, whole blood, and biopsy samples of body tissue. This disclosure is drawn, inter alia, to methods of diagnosing and monitoring proliferative hematological disorders using profiles of the ubiquitin-proteasome system (UPS).

The ubiquitin-proteasome system (UPS) plays a major role in the most important processes that control cell homeostasis in normal and neoplastic states. The present inventors have discovered that analyzing various components of the UPS can provide a profile that may be used for classifying and stratifying cancer patients for diagnosis, therapy, and prediction of clinical behavior.

In the context of cancer diagnosis, it is frequently difficult to have access to the diseased cells. This is true even in hematologic diseases because of the problem of dilution effects by normal cells. In various embodiments, the present methods overcome problems of cancer diagnosis by determining the levels of proteasomes and proteasomal peptidase activities in the plasma of patients with proliferating hematological disorders. By studying the levels of proteasome, ubiquitin, and proteasome enzymatic activities in the plasma, a UPS profile of the leukemic blasts can be determined. Analysis of the UPS profile reveals that leukemic cells have higher number of proteasomes, but the specific enzymatic activities is, in general, lower than the specific activity of mature hematopoietic cells. Moreover, the UPS profiles of subjects with different hematopoietic disorders are distinct, which allow for an accurate diagnosis of disease. For example, ALL has a different UPS profile than that of AML and both have a different profile from MDS. In addition, correlation with clinical behavior is different depending on the disease. The use of UPS profiles in diagnosing proliferative hematological disorders is described in further detail below and in the Examples.

In some embodiments, the methods may be used to generate a UPS profile which can predict survival of a subject having a proliferative hematological disorder. For instance, the ability of a UPS profile to predict survival within the intermediate cytogenetic group, as well as within the unfavorable cytogenetic group, has particular clinical importance. These patient groups are difficult to manage, and profiling proteasome protein and activity using plasma is useful for the selection of appropriate treatments. In addition, testing for the specific activity of Cas-L/p may be useful to identify patients with ALL who will have short remission duration.

In one aspect, the methods generally provide for the detection, measuring, and comparison of a pattern of UPS proteins and/or activities in a patient sample. Accordingly, the various aspects relate to the collection, preparation, separation, identification, characterization, and comparison of the abundance of UPS proteins and/or activities in a test sample. The technology further relates to detecting and/or monitoring a sample containing one or more UPS proteins or activities, which are useful, alone or in combination, to determine the presence or absence of a proliferative hematological disorder or any progressive state thereof.

### Sample Preparation

Test samples of acellular body fluid or cell-containing samples may be obtained from an individual or patient. Methods of obtaining test samples are well-known to those of skill in the art and include, but are not limited to, aspirations or drawing of blood or other fluids. Samples may include, but are not limited to, whole blood, serum, plasma, saliva, cerebrospinal fluid (CSF), pericardial fluid, pleural fluid, urine, and eye fluid.

In embodiments in which the proteasome activity will be determined using an acellular body fluid, the test sample may be a cell-containing liquid or an acellular body fluid (e.g., plasma or serum). In some embodiments in which the test sample contains cells, the cells may be removed from the liquid portion of the sample by methods known in the art (e.g., centrifugation) to yield acellular body fluid for the proteasome activity measurement. In suitable embodiments, serum or plasma are used as the acellular body fluid sample. Plasma and serum can be prepared from whole blood using suitable methods well-known in the art. In these embodiments, data may be normalized by volume of acellular body fluid.

In some embodiments, the proteasomal peptidase activity is determined using a cell-containing sample. In these embodiments, the cell-containing sample includes, but is not limited to, blood, urine, organ, and tissue samples. In suitable embodiments, the cell-containing sample is a blood sample, such as a blood cell lysate. Cell lysis may be accomplished by standard procedures. In certain embodiments, the cell-containing sample is a whole blood cell lysate. Kahn et al. (Biochem. Biophys. Res. Commun., 214:957-962 (1995)) and Tsubuki et al. (FEBS Lett., 344:229-233 (1994)) disclose that red blood cells contain endogenous proteinaceous inhibitors of the proteasome. However, endogenous proteasomal peptidase inhibitors can be inactivated in the presence of SDS at a concentration of about 0.05%, allowing red blood cell lysates and whole blood cell lysates to be assayed reliably. At this concentration of SDS, most if not all proteasomal peptidase activity is due to the 20S proteasome. Although purified 20S proteasome exhibits poor stability at 0.05% SDS, 20S proteasomal peptidase activity in cell lysates is stable under these conditions (Vaddi et al., U.S. Pat. No. 6,613,541).

In other embodiments, the cell-containing sample is a white blood cell lysate. Methods for obtaining white blood cells from blood are known in the art (Rickwood et al., Anal. Biochem., 123;23-31 (1982); Fotino et al., Ann. Clin. Lab. Sci., 1:131 (1971)). Commercial products useful for cell separation include without limitation Ficoll-Paque (Pharmacia Biotech) and NycoPrep (Nycomed). In some situations, white blood cell lysates provide better reproducibility of data than do whole blood cell lysates.

Variability in sample preparation of cell-containing samples can be corrected by normalizing the data by, for example, protein content or cell number. In certain embodiments, proteasomal peptidase activity in the sample may be normalized relative to the total protein content or proteasomal protein content in the sample (specific activity method). Total protein content in the sample can be determined using standard procedures, including, without limitation, Bradford assay and the Lowry method. In other embodiments, proteasomal peptidase activity in the sample may be normalized relative to cell number.

### Measuring Proteasome Level

In one embodiment, the quantity or concentration proteasomal s may be measured by determining the amount of one or more proteasomal proteins in a sample. The polypeptides in the proteasome can be detected by an antibody which is detectably labeled, or which can be subsequently labeled. A variety of formats can be employed to determine whether a sample contains a proteasomal protein or proteins that bind to a given antibody. Immunoassay methods useful in the detection of proteasomal proteins include, but are not limited to, e.g., dot blotting, western blotting, protein chips, immunoprecipitation (IP), competitive and non-competitive protein binding assays, enzyme-linked immunosorbent assays (ELISA), and others commonly used and widely-described in scientific and patent literature, and many employed commercially.

Proteins from samples can be isolated using techniques that are well-known to those of skill in the art. The protein isolation methods employed can, e.g., be including, but not limited to, e.g., those described in Harlow & Lane, Antibodies: A Laboratory Manual (Cold Spring Harbor Laboratory Press, Cold Spring Harbor, N.Y., 1988). In some embodiments, proteasomal protein is extracted from the acellular body fluid sample. Plasma purification methods are known in the art such. See e.g., Cohn, E. J., et al., Am. Chem. Soc., 62:3396-3400.(1940); Cohn, E. J., et al., J. Am. Chem. Soc., 72:465-474 (1950); Pennell, R. B., Fractionation and isolation of purified components by precipitation methods, pp. 9-50. In The Plasma Proteins, Vol. 1, F. W. Putman (ed.). Academic Press, New York (1960); and U.S. Pat. No. 5,817,765.

Antibodies can be used in methods, including, but not limited to, e.g., western blots or ELISA, to detect the expressed protein complexes. In such uses, it is possible to immobilize either the antibody or proteins on a solid support. Supports or carriers include any support capable of binding an antigen or an antibody. Well-known supports or carriers include, but arc not limited to, e.g., glass, polystyrene, polypropylene, polyethylene, dextran, nylon, amylases, natural and modified celluloses, polyacrylamides, gabbros, and magnetite.

Antibodies may be specific for one or more proteins that comprise the proteasomal complex. In one embodiment, the quantity or concentration of proteasomes in a sample is determined by detecting the quantity or concentration of one or more proteins that interact to form the proteasomal complex. In one embodiment, the quantity or concentration of proteasomes in a sample is determined using a polyclonal antibody to the 20S Proteasome core subunits. In other embodiments, the quantity or concentration of proteasomes in a sample is determined using a polyclonal or a monoclonal antibody directed to one or more proteins including, but not limited to, Ki-67 protein, 19S Regulator ATPase Subunit Rpt4; 19S Proteasome S5A-Subunit; 19S Proteasome S5A-Subunit,; 19S Proteasome, S6-Subunit; 20S Proteasome α1, 2, 3, 5, 6, & 7-Subunits; 20S Proteasome α1-Subunit; 20S Proteasome α3-Subunit; 20S Proteasome α5-Subunit; 20S Proteasome α7-Subunit; 20S Proteasome β1-Subunit; 20S Proteasome β3-Subunit; 20S Proteasome β4-Subunit; 20S Proteasome β5i-Subunit; 26S Proteasome S4-Subunit; 26S Proteasome, S7-Subunit; Proteasome Activator PA700 Subunit 10B; 19S Regulator ATPase Subunit Rpt1; and 19S Regulator non-ATPase Subunit Rpn10.

Methods of generating antibodies are well known in the art, see, e.g., Sambrook, et al., 1989, Molecular Cloning; A Laboratory Manual, Second Edition, Cold Spring Harbor Press, Plainview, N.Y. Antibodies may be detectably labeled by methods known in the art. Labels include, but are not limited to, radioisotopes such as ³H, ¹⁴C, ³⁵S, ³²P, ¹²³I, ¹²⁵I, ¹³¹I), enzymes (e.g., peroxidase, alkaline phosphatase, beta-galactosidase, luciferase, alkaline phosphatase, acetylcholinesterase and glucose oxidase), enzyme substrates, luminescent substances (e.g., luminol). fluorescent substances (e.g., FITC, rhodamine, lanthanide phosphors), biotinyl groups (which can be detected by marked avidin e.g., streptavidin containing a fluorescent marker or enzymatic activity that can be detected by optical or colorimetric methods), predetermined polypeptide epitopes recognized by a secondary reporter (e.g., leucine zipper pair sequences, binding sites for secondary antibodies, metal binding domains, epitope tags) and colored substances. In binding these labeling agents to the antibody, the maleimide method (Kitagawa, T., et al., J. Biochem., 79:233-236 (1976)), the activated biotin method (Hofmann, K., et al., J. Am. Chem. Soc., 100:3585 (1978)) or the hydrophobic bond method, for instance, can be used.

In some embodiments, labels are attached via spacer arms of various lengths to reduce potential steric hindrance. Antibodies may also be coupled to electron dense substances, such as ferritin or colloidal gold, which are readily visualized by electron microscopy.

Where a radioactive label is used as a detectable substance, proteins may be localized by autoradiography. The results of autoradiography may be quantitated by determining the density of particles in the autoradiographs by various optical methods, or by counting the grains.

The antibody or sample may be immobilized on a carrier or solid support which is capable of immobilizing cells, antibodies, etc. For example, the carrier or support may be nitrocellulose, or glass, polyacrylamides, gabbros, and magnetite. The support material may have any possible configuration including spherical (e.g. head), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against one or more proteins that comprise a proteasome. Antibodies to proteasomal proteins are available commercially through multiple sources. For example, polyclonal antibodies directed to proteasome core subunit are available from Biomol International, Cat. No. PW8155-0100 (Plymouth, Pa.). Monoclonal antibodies directed to proteasome α subnit are available from Biomol International, Cat. No. PW8100 (Plymouth, Pa.).

Immunoassays, or assays to detect an antigen using an antibody, are well known in the art and can take many forms, e.g., radioimmunoassay, immunoprecipitation, Western blotting, enzyme-linked immunosorbent assay (ELISA), and 2-site or sandwich immunoassay.

In one embodiment, a sandwich ELISA is used. In this assay, two antibodies to different segments, or epitopes, of the antigen are used. The first antibody (capture antibody) is coupled to a solid support. When a sample of bodily fluid is contacted with the capture antibody on the solid support, the antigen contained in the bodily fluid is captured on the solid support through a specific interaction between antigen and antibody, resulting in the formation of a complex. Washing of the solid support removes unbound or non-specifically bound antigen. Subsequent exposure of the solid support to a detectably-labeled second antibody (detection antibody) to the antigen (generally to a different epitope than the capture antibody) enables the detection of bound or captured antigen. As would be readily recognized by one of skill in the art, assaying additional markers in parallel to assaying for proteasomal protein is possible with the use of distinct pairs of specific antibodies, each of which is directed against a different marker.

In an illustrative embodiment, a electro-chemiluminescent sandwich immunoassay is used. In this assay, two antibodies to different segments, or epitopes, of the antigen are used. For instance, antibody to one or more proteasomal proteins is coated on plates to capture the proteasomes. The antibody may be a mouse monoclonal antibody to proteasome alpha subunit. A sample is contacted to the plate, and after incubation under appropriate binding conditions, the plate is washed. After the wash, primary detection antibody, which binds to the one or more proteasomal proteins, is added to each well and incubated. After another wash, a Sulfo-tag labeled secondary antibody (capable of binding to the primary antibody) is added to each well and incubated for another hour. After a final wash, a MSD read buffer is added and signal is detected by MSD Sector2400 (MSD, Gaithersburg, Md.).

Relative or actual amounts of proteasomes in body fluids can be determined by methods well known in the art. See, e.g., Drach, J., et al., Cytometry, 10(6):743-749 (1989). For example, a standard curve can be obtained using known amounts of proteasomes, i.e., proteasome standards. The actual amount of the proteasomes in a body fluid may thus be determined using the standard curve. Another approach that does not use a standard curve is to determine the dilution of body fluid that gives a specified amount of signal. The dilution at which 50% of the signal is obtained is often used for this purpose. In this case, the dilution at 50% maximal binding of proteasomes in a patient body fluid is compared with the dilution at 50% of maximal binding for proteasomes obtained in the same assay using a reference sample (i.e., a sample taken from the corresponding bodily fluid of normal individuals, free of proliferative disorders).

Monoclonal or polyclonal antibodies may be used as the capture and detection antibodies in sandwich immunoassay systems. Monoclonal antibodies are specific for single epitope of an antigen and allow for detection and quantitation of small differences in antigen. Polyclonal antibodies can he used as the capture antibody to capture large amounts of antigen or can be used as the detection antibody. A monoclonal antibody can be used as the either the capture antibody or the detection antibody in the sandwich assay to provide greater specificity. In some embodiments, polyclonal antibodies are used as the capture antibody and monoclonal antibodies are used as the detection antibody.

One consideration in designing a sandwich ELISA is that the capture and detection antibodies should be generated against or recognize “non-overlapping” epitopes. The phrase “non-overlapping” refers to epitopes, which are segments or regions of an antigen that are recognized by an antibody, that arc sufficiently separated from each other such that an antibody for each epitope can hind simultaneously. That is, the binding of one antibody (e.g., the capture antibody) to a first epitope of the antigen should not interfere with the binding of a second antibody (e.g., the detection antibody) to a second epitope of the same antigen. Capture and detection antibodies that do not interfere with one another and can bind simultaneously are suitable for use in a sandwich ELISA.

Methods for immobilizing capture antibodies on a variety of solid surfaces arc well-known in the art. The solid surface may be composed of any of a variety of materials, for example, glass, quartz, silica, paper, plastic, nitrocellulose, nylon, polypropylene, polystyrene, or other polymers. The solid support may be in the form of beads, microparticles, microspheres, plates which arc flat or comprise wells, shallow depressions, or grooves, microwell surfaces, slides, chromatography columns, membranes, filters, or microchips. In one embodiment, the solid support is a microwell plate in which each well comprises a distinct capture antibody to a specific marker so that multiple markers may be assayed on a single plate. In another embodiment, the solid support is in the form of a bead or microparticle. These beads may be composed of, for example, polystyrene or latex. Beads may be of a similar size or may be of varying size. Beads may be approximately 0.1 μm-10 μm in diameter or may be as large as 50 μm-100 μm in diameter.

Methods of identifying the binding of a specific binding agent to proteasomes are known in the art and vary dependent on the nature of the label. In suitable embodiments, the detectable label is a fluorescent dye. Fluorescent dyes arc detected through exposure of the label to a photon of energy of one wavelength, supplied by an external source such as an incandescent lamp or laser, causing the fluorophore to be transformed into an excited state. The fluorophore then emits the absorbed energy in a longer wavelength than the excitation wavelength which can be measured as fluorescence by standard instruments containing fluorescence detectors. Exemplary fluorescence instruments include spectrofluorometers and microplate readers, fluorescence microscopes, fluorescence scanners, and flow cytometers.

In one embodiment, a sandwich assay is constructed in which the capture antibody is coupled to a solid support such as a head or microparticle. Captured antibody-antigen complexes, subsequently bound to detection antibody, are detected using flow cytometry and is well-known in the art. Flow cytometers hydrodynamically focus a liquid suspension of particles (e.g., cells or synthetic microparticles or beads) into an essentially single-file stream of particles such that each particle can be analyzed individually. Flow cytometers are capable of measuring forward and side light scattering which correlates with the size of the particle. Thus, particles of differing sizes or fluorescent characteristics may be used in invention methods simultaneously to detect distinct markers. Fluorescence at one or more wavelengths can be measured simultaneously. Consequently, particles can be sorted by size and the fluorescence of one or more fluorescent labels can be analyzed for each particle. Exemplary flow cytometers include the Becton-Dickinson Immunocytometry Systems FACSCAN. Equivalent flow cytometers can also be used in the invention methods.

### Measuring Proteasome Activity

Proteasome activity in the test sample can be measured by any assay method suitable for determining 20S or 26S proteasome peptidase activity. (See, e.g., Vaddi et al., U.S. Pat. No. 6,613,541; McCormack et al., Biochemistry, 37:7792-7800 (1998)); Driscoll and Goldberg, J. Biol. Chem., 265:4789 (1990); Orlowski et al., Biochemistry, 32:1563 (1993)). In a suitable embodiment, a substrate having a detectable label is provided to the reaction mixture and proteolytic cleavage of the substrate is monitored by following disappearance of the substrate or appearance of a cleavage product. Detection of the label may be achieved, for example, by fluorometric, colorimetric, or radiometric assay.

Substrates for use in determining proteasomal peptidase activity may be chosen based on the selectivity of each peptidase activity. For example, the chymotrypsin-like peptidase preferentially cleaves peptides on the carboxyl side of tyrosine, tryptophan, phenylalanine, leucine, and methionine residues. The trypsin-like peptidase preferentially cleaves peptides on the carboxyl side of arginine and lysine residues. The caspase-like peptidase (or peptidylglutamyl-peptide hydrolase) preferentially cleaves peptides at glutamic acid and aspartic acid residues. Based on these selectivities, the skilled artisan can choose a specific substrates for each peptidase.

Suitable substrates for determining 26S proteasome activity include, without limitation, lysozyme, α-lactalbumin, β-lactoglobulin, insulin b-chain, and ornithine decarboxylase. When 26S proteasome activity is to be measured, the substrate is typically ubiquitinated or the reaction mixture further contains ubiquitin and ubiquitination enzymes.

In some embodiments, the substrate is a peptide less than 10 amino acids in length. In one embodiment, the peptide substrate contains a cleavable fluorescent label and release of the label is monitored by fluorometric assay. Non-limiting examples of substrates to measure trypsin-like activity include N-(N-benzoylvalylglycylarginyl)-7-amino-4-methylcoumarin (Bz-Val-Gly-Arg-AMC), N—(N-carbobenzyloxycarbonylleucylleucylarginyl)-7-amino-4-methylcoumarin (Z-Leu-Leu-Arg-AMC), Ac-Arg-Leu-Arg-AMC, and Boc-Leu-Arg-Arg-AMC. Non-limiting examples of substrates to measure caspase-like activity include N—(N-carbobenzyloxycarbonylleucylleucylglutamyl)-2-naphthylamine (Z-Leu-Leu-Glu-2NA). N—(N-carbobenzyloxycarbonylleucylleucylglutamyl)-7-amino-4-methylcoumarin (Z-Leu-Leu-Glu-AMC), and acetyl-L-glycyl-L-prolyl-L-leucyl-L-aspartyl-methylcoumarin (Ac-Gly-Pro-Leu-Asp-AMC). Non-limiting examples of substrates to measure chymotrypsin-like activity include N—(N-succinylleucylleucylvalyltyrosyl)-7-amino-4-methylcoumarin (Suc-Leu-Leu-Val-Tyr-AMC), Z-Gly-Gly-Leu-2NA. Z-Gly-Gly-Leu-AMC, and Suc-Arg-Pro-Phe-His-Leu-Leu-Val-Tyr-AMC.

Suitable substrates for measuring the chymotrypsin-like, caspase-like, and trypsin-like activities of the proteasome are Suc-Leu-Leu-Val-Tyr-AMC, Z-Leu-Leu-Glu-AMC, and Bz-Val-Gly-Arg-AMC, respectively, and the release of the cleavage product, AMC, can be monitored at 440 nm (λₑₓ=380 nm). Cleavage due to a particular peptidase may be determined by, for example, using a substrate specific for that peptidase and assaying that activity independent of other peptidases.

In certain embodiments, the reaction mixture further contains a 20S proteasome activator. Activators include those taught in Coux et al. (Ann. Rev. Biochem., 65:801-847 (1995)), such as PA28 or sodium dodecyl sulfate (SDS). However, SDS is not compatible with Bz-Val-Gly-Arg-AMC, therefore when Bz-Val-Gly-Arg-AMC is chosen as the substrate, PA28 is used instead of SDS to activate the proteasome.

### Diagnosis of Disease States

In some embodiments, the specific activity level of one or more proteasomal peptidases (e.g., Ch-L/p, Tr-L/p, and Cas-L/p) in a test sample is used to diagnose a disease. In these embodiments, the level of proteasome activity measured in the test sample is normalized to the level of one or more proteasomal proteins to provide a specific activity value for the one or more proteasomal peptidases. The specific activity value may be compared to a reference value to determine if the levels of specific activity arc elevated or reduced relative to the reference value. Typically, the reference value is the specific activity measured in a comparable sample from one or more healthy individuals. An increase or decrease in the specific activity may be used in conjunction with clinical factors other than proteasomal peptidase activity to diagnose a disease.

Association between a pathological state (e.g., a proliferative hematological disorder) and the aberration of a specific activity level of one or more proteasomal peptidases can be readily determined by comparative analysis in a normal population and an abnormal or affected population. Thus, for example, one can study the specific activity level of one or more proteasomal peptidases in both a normal population and a population affected with a particular pathological state. The study results can be compared and analyzed by statistical means. Any detected statistically significant difference in the two populations would indicate an association. For example, if the specific activity is statistically significantly higher in the affected population than in the normal population, then it can be reasonably concluded that higher specific activity is associated with the pathological state.

Statistical methods can be used to set thresholds for determining when the specific activity level in a subject can be considered to be different than or similar to a reference level. In addition, statistics can be used to determine the validity of the difference or similarity observed between a patient's specific activity level and the reference level. Useful statistical analysis methods are described in L. D. Fisher & G. vanBelle, Biostatistics: A Methodology for the Health Sciences (Wiley-Interscience, NY, 1993). For instance, confidence (“p”) values can be calculated using an unpaired 2-tailed t test, with a difference between groups deemed significant if the p value is less than or equal to 0.05. As used herein a “confidence interval” or “CI” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Preferred confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Preferred p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistics for Research, John Wiley & Sons, New York, 1983. Exemplary statistical tests for associating a prognostic indicator with a predisposition to an adverse outcome are described hereinafter.

Once an association is established between a specific activity and a pathological state, then the particular physiological state can be diagnosed or detected by determining whether a patient has the particular aberration, i.e. elevated or reduced specific activity levels.

The term “elevated levels” or “higher levels” as used herein refers to levels of a specific activity that are higher than what would normally be observed in a comparable sample from control or normal subjects (i.e., a reference value). In some embodiments, “control levels” (i.e., normal levels) refer to a range of specific activity levels that would be normally be expected to be observed in a mammal that does not have a proliferative hematological disorder. A control level may he used as a reference level for comparative purposes. “Elevated levels” refer to specific activity levels that are above the range of control levels. The ranges accepted as “elevated levels” or “control levels” are dependent on a number of factors. For example, one laboratory may routinely determine the specific activity of an enzyme in a sample that are different than the specific activity obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated values” of the present invention. For example, a series of samples from control subjects and subjects diagnosed with proliferative hematological disorders can be used to establish ranges that are “normal” or “control” levels and ranges that are “elevated” or “higher” than the control range.

Similarly, “reduced levels” or “lower levels” as used herein refer to levels of a peptidase specific activity that are lower than what would normally be observed in a comparable sample from control or normal subjects (i.e., a reference value). In some embodiments. “control levels” (i.e. normal levels) refer to a range of specific activity levels that would be normally be expected to be observed in a mammal that does not have a proliferative hematological disorder and “reduced levels” refer to proteasome activity levels that are below the range of such control levels.

For example, elevated specific activity of Cas-L/p and/or Tr-L/p as compared to the corresponding reference values from healthy individuals are associated with the presence of MDS. While an elevation in any of these specific activities alone may not provide a conclusive diagnosis, the information is useful in conjunction clinical factors other than specific activity commonly used in the diagnosis of, for example, leukemia; the specific activity then provides a further factor in confirming a diagnosis.

Moreover, reduced specific activity of Ch-L/p as compared to the corresponding reference values from healthy individuals is associated with the presence of AML or ALL. A reduced specific activity of Cas-L/p and/or Tr-L/p as compared to the corresponding reference values from healthy individuals is associated with the presence of ALL.

The specific activity level of one or more peptidases in a test sample can be used in conjunction with clinical factors other than specific activity to diagnose a disease. Clinical factors of particular relevance in the diagnosis of proliferative hematological disorders include, but are not limited to, the patient's medical history, a physical examination of the patient, complete blood count, examination of bone marrow cells, cytogenetics, and immunophenotyping of blood cells.

Monitoring Progression and/or Treatment

In one aspect, the specific activity level of one or more proteasomal peptidases (e.g., Ch-L/p, Tr-L/p, and Cas-L/p) in a biological sample of a patient is used to monitor the effectiveness of treatment or the prognosis of disease. In some embodiments, the specific activity level of one or more proteasomal peptidases in a test sample obtained from a treated patient can be compared to the level from a reference sample obtained from that patient prior to initiation of a treatment. Clinical monitoring of treatment typically entails that each patient serve as his or her own baseline control. In some embodiments, test samples are obtained at multiple time points following administration of the treatment. In these embodiments, measurement of specific activity level of one or more proteasomal peptidases in the test samples provides an indication of the extent and duration of in vivo effect of the treatment.

### Determining Prognosis

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

Additionally, a change in a clinical factor from a baseline level may impact a patient's prognosis, and the degree of change in level of the clinical factor may be related to the severity of adverse events. Statistical significance is often determined by comparing two or more populations, and determining a confidence interval and/or a p value.

Multiple determinations of proteasomal specific activity levels can be made, and a temporal change in activity can be used to determine a prognosis. For example, comparative measurements are made of the specific activity of an acellular body fluid in a patient at multiple time points, and a comparison of a specific activity value at two or more time points may he indicative of a particular prognosis.

A prognosis is often determined by examining one or more clinical factors and/or symptoms that correlate to patient outcomes. As described herein, the specific activity level of a proteasomal peptidase is a clinical factor useful in determining prognosis. The skilled artisan will understand that associating a clinical factor with a predisposition to an adverse outcome may involve statistical analysis.

In certain embodiments, the levels of specific activity of one or more proteasomal peptidases are used as indicators of an unfavorable prognosis. According to the method, the determination of prognosis can be performed by comparing the measured specific activity level to levels determined in comparable samples from healthy individuals or to levels known to corresponding with favorable or unfavorable outcomes. The absolute specific activity levels obtained may depend on an number of factors, including, but not limited to, the laboratory performing the assays, the assay methods used, the type of body fluid sample used and the type of disease a patient is afflicted with. According to the method, values can be collected from a series of patients with a particular disorder to determine appropriate reference ranges of specific activity for that disorder. One of ordinary skill in the art is capable of performing a retrospective study that compares the determined specific activity levels to the observed outcome of the patients and establishing ranges of levels that can be used to designate the prognosis of the patients with a particular disorder. For example, specific activity levels in the lowest range would he indicative of a more favorable prognosis, while specific activity levels in the highest ranges would be indicative of an unfavorable prognosis. Thus, in this aspect the term “elevated levels” refers to levels of specific activity that are above the range of the reference value. In some embodiments patients with “high” or “elevated” specific activity levels have levels that are higher than the median activity in a population of patients with that disease. In certain embodiments, “high” or “elevated” specific activity levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder.

Because the level of specific activity in a test sample from a patient relates to the prognosis of a patient in a continuous fashion, the determination of prognosis can be performed using statistical analyses to relate the determined specific activity levels to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses.

In certain embodiments, the prognosis of ALL, AML, CLL, or MDS patients can be correlated to the clinical outcome of the disease using the specific activity level and other clinical factors. Simple algorithms have been described and are readily adapted to this end. The approach by Giles et. al., British Journal of Hemotology, 121:578-585, is exemplary. As in Giles et al., associations between categorical variables (e.g., proteasome activity levels and clinical characteristics) can be assessed via crosstabulation and Fisher's exact test. Unadjusted survival probabilities can be estimated using the method of Kaplan and Meier. The Cox proportional hazards regression model also can be used to assess the ability of patient characteristics (such as proteasome activity levels) to predict survival, with ‘goodness of fit’ assessed by the Grambsch-Therneau test, Schoenfeld residual plots, martingale residual plots and likelihood ratio statistics (see Grambsch et al, 1995). In some embodiments, this approach can be adapted as a simple computer program that can be used with personal computers or personal digital assistants (PDA). The prediction of patients' survival time in based on their proteasome activity levels can be performed via the use of a visual basic for applications (VBA) computer program developed within Microsoft® Excel. The core construction and analysis may be based on the Cox proportional hazard models. The VBA application can he developed by obtaining a base hazard rate and parameter estimates. These statistical analyses can be performed using a statistical program such as the SAS® proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner.

In some embodiments of the invention, multiple prognostic factors, including specific activity level, are considered when determining the prognosis of a patient. For example, the prognosis of an AML, ALL, CLL, or MDS patient may be determined based on specific activity and one or more prognostic factors selected from the group consisting of cytogenetics, performance status, AHD (antecedent hematological disease), age, and diagnosis (e.g., MDS v. AML). In certain embodiments, other prognostic factors may be combined with the specific activity level in the algorithm to determine prognosis with greater accuracy.

### Predicting Response to Therapy

In one aspect, the specific activity level of one or more proteasomal peptidases in a patient can be used to predict response to therapy for patients having a proliferative hematological disorder of a particular risk category according to cytogenetic analysis (e.g., ISCN standards). Cytogenetics refers to the analysis of the physical appearance of chromosomes (e.g., the number and shape of the chromosomes). The identification of particular chromosome alterations or abnormalities can be helpful in diagnosing, for example, specific types of leukemia and lymphoma. Furthermore, particular types of chromosomal alterations have been associated with clinical behavior or response to therapy and therefore can be used in determining treatment approaches. For example, patients with AML are assigned to one of several risk categories (i.e., good, intermediate, bad, and very had) based on the appearance of metaphase chromosomes according to the International System for Cytogenetic Nomenclature (ISCN). Thus, in AML, patients in the good-risk category or having good cytogenetics exhibit t(8;21) or inv16; intermediate-risk or intermediate cytogenetics exhibit a normal karyotype (NN) or −Y only; the bad-risk category or had cytogenetics include all other abnormalities, without good and very bad cytogenetic features; and very bad risk −5, 5q-, −7, 7q-, complex abnormalities (i.e., clones with a number of unrelated abnormalities), abnormal (abn) 3q, t(9;22), t(6;9), or abn 11q23 and absence of good cytogenetic features. For example, AML patients having good cytogenetics or in the good-risk category are treated with chemotherapy, while patients having had or very had cytogenetics or are in the highest risk categories are treated with bone marrow transplants. However, approximately half of AML patients have “normal” cytogenetics and therefore fall into an intermediate risk group, wherein the treatment and the response thereto can vary considerably (Marcucci et al., Curr Opin Hematol, 12(1):68-75, 2005).

### Kits

A kit may be used for conducting the diagnostic and prognostic methods described herein. Typically, the kit should contain, in a carrier or compartmentalized container, reagents useful in any of the above-described embodiments of the diagnostic method. The carrier can be a container or support, in the form of, e.g., bag, box, tube, rack, and is optionally compartmentalized. The carrier may define an enclosed confinement for safety purposes during shipment and storage. In one embodiment, the kit includes an antibody selectively immunoreactive with a proteasome. The antibodies may be labeled with a detectable marker such as radioactive isotopes, or enzymatic or fluorescence markers. Alternatively, secondary antibodies such as labeled anti-IgG and the like may be included for detection purposes. In addition, reagents to detect the activity of one or more proteasomal peptidases may be provided. Optionally, the kit can include standard proteasomes prepared or purified for comparison purposes. Instructions for using the kit or reagents contained therein are also included in the kit.

### EXAMPLES

The present methods and kits, thus generally described, will be understood more readily by reference to the following examples, which arc provided by way of illustration and are not intended to he limiting of the present methods and kits. The following is a description of the materials and experimental procedures used in the example.

### Example 1

### UPS Profiling in Patients with AML, ALL, and MDS

### Materials and Methods

Patients and Samples. All samples from patients and healthy volunteers were collected under an internal review board-approved protocol with written informed consent. Patient samples were collected during the period 2001-2003 without selection prior to initiating therapy at MD Anderson Cancer Center (Houston, Tex.). All patients were newly diagnosed, but the majority were referred after diagnosis by their local physician within a few days of their diagnosis. Diagnosis of AML and advanced MDS was made at MD Anderson based on blood counts, flow cytometry, and molecular studies performed on peripheral blood and bone marrow samples. Plasma was separated from EDTA peripheral blood tubes by centrifuging at 1500×g for 10 min at 4° C. Plasma samples obtained from apparently healthy volunteers were used as controls for each study. Plasma samples were stored at −70° C. until analysis. Both AML and MDS patients were treated at MD Anderson with standard therapy based on idarubicine+ ara-C with minor variations (±topotecan or fludarabine). All patients with MDS had advanced disease and were candidates for chemotherapy. Advanced MDS disease is defined by the presence of severe anemia (hemoglobin <8 g/dL), thrombocytopenia (<50×10⁹/L platelet count), or >10% blasts.

Measurement of Proteasome Protein Levels. For proteasome quantitation in human plasma, a sandwich immunoassay based on electro-chemiluminescence technology (MSD, Gaithersburg, Md.) was used. Monoclonal antibody (MCP20, Biomol Cat. No. PW8100, Plymouth, Pa.) was coated on MSD goat anti-mouse (GAM) plates to capture proteasome alpha subunit. Proteasome standards (range, 0.1-400 ng/mL) (Biomol International. Cat. No. PW8720), controls, and patient plasma samples were all diluted 1:20 in MSD lysis buffer and added to each well. After incubation at room temperature (RT) for 2 hours, the plate was washed. After the wash, detection antibody (polyclonal, anti-core subunit, Biomol International, Cat. No. PW8155-0100) was added to each well and incubated for an hour. After another wash, the Sulfo-tag labeled goat anti-rabbit (GAR) antibody was added to each well and incubated for another hour. After a final wash, the MSD read buffer was added and signal was detected by MSD Sector2400. The proteasome level (ng/mL) was calculated using the proteasome standard curve.

Measurement of Ubiquitin Protein. The level of ubiquitin protein (poly-ubiquitin) in plasma was quantitated by a sandwich immunoassay also using electro-chemiluminescence technology. Briefly, after 2 h blockage of the MSD GAM plate, an anti-ubiquitin monoclonal antibody (clone FK1, Biomol, Cat. No. PW8805) was coated overnight onto the MSD GAM single spot plate at 4° C. on a shaker. Ubiquitin positive control (Cat. No. 89899, Pierce, Rockford, Ill.) was used as a calibrator to create a 7 point standard curve using Hel cell lysate. Plasma samples were diluted 1:2 using MSD lysis buffer. Calibrator, reference standards, and plasma samples were added to the plate and incubated for 3 hours on a shaker at room temperature. In this incubation, ubiquitin (poly-ubiquitin) was specifically captured with the anti-polyubiquitin. After washing, Sulfo-tag labeled anti-ubiquitin was added to the plate and incubated for 1 h. After a final wash, the MSD read buffer was added to the plate and signal was read on the MSD Sector2400. The ubiquitin levels were determined by reading against a standard curve and converting to ng ubiquitin/mL plasma.

Measurement of Proteasome Enzymatic Activities. A fluorogenic kinetic assay using peptide-AMC (7-amino 4-methylcoumarin) substrates was used to measure the Ch-L, Tr-L, and Cas-L activities in the plasma. Briefly, Ch-L, Cas-L, and Tr-L activities were assayed by continuously monitoring the production of 7-amino-4-methylcoumarin (AMC) from 3 separate fluorogenic peptides. Plasma (45 μL) was first mixed with 5 μL 10% SDS at room temperature for 15 min to activate the plasma. The reaction wells contained 30 μL assay buffer (0.05% SDS in 25 mM HEPES), 10 μL activated plasma, and 10 μL of the fluorogenic peptide-AMC substrate.

Substrate working solutions (1 mM) were made by adding 950 μL buffer A (1× HEPES/0.05% SDS) to 50 μL stock solution of Ch-L and Cas-L substrates (20 mM Sue-LLVY-AMC and 20 mM Z-LLE-AMC) and mixed. 950 μL buffer B (1× HEPES/0.05% Tween 20) was added to 50 μL stock solution of Tr-L substrate (20 mM BZ-VGR-AMC) and mixed. Working solutions were protected from light. A microwell plate was set-up as follows: 30 μL buffer A was pipetted into the wells of columns 1, 4, 7, and 10 (to measure Ch-L activity) and 3, 6, 9, and 12 (to measure Cas-L activity); 30 μL buffer B was pipetted into the wells of columns 2, 5, 8, and 11 (to measure Tr-L); 10 μL buffer A or B served as blank controls. Next, 10 μL of processed samples were pipetted into duplicate wells for each substrate (i.e., 6 wells per processed plasma sample), to the designated wells according to the plate map. Ten (10) μL of substrate was added to the designated wells according to the plate map. The final concentration of substrate was 200 μM. Cleavage of substrate was detected using the SPECTRAmax GEMINI EM instrument with SoftMax Pro data Collection software. The instrument incubation chamber temperature was set to 37° C. Fluorescence excitation and emission wavelengths were 380 nm and 460 nm, respectively. Samples were read at 1 min intervals over 30 min. Green fluorescence represented Ch-L activity; blue fluorescence represented Cas-L activity; and yellow fluorescence represented Tr-L activity. Enzymatic activities were quantitated by generating a standard curve of AMC (range, 0-8 μM). The slope of the AMC standard curve was used as a conversion factor to calculate the 3 enzymatic activities for each sample as pmol AMC/sec/mL plasma. The specific activity of each proteasomal peptidases (Ch-L/p, Tr-L/p, and Cas-L/p) was normalized to the amount of proteasomes in the sample and expressed as pmol AMC/sec/pg proteasome.

Statistical Methods. Clinical and biological characteristics were analyzed for their association with response and survival using log-rank test and multivariate Cox proportional hazards models (Cox, J Royal Stat Soc, 34:187-220 (1972)). Estimates of survival curves from the time of initiating therapy were calculated according to the Kaplan-Meier product-limit method (Kaplan and Meier, J. American Statistical Association, 53:457-481 (1958)). Univariate and multivariate Cox proportional hazard models were developed; predictive variables with P values of less than 0.10 for the univariate Cox proportional hazards model were included in a multivariate model.

High Levels of Circulating Proteasome and Ubiquitin Occur in Patients with Acute Leukemia

Complete clinical data for AML, MDS, and ALL patients were recorded at the time of diagnosis prior to initiating therapy (Table 1). Patients with advanced MDS were treated with AML therapy. Few AML patients had good cytogenetics [inv16, t(8;21), or t(15;17)] and about one-third had unfavorable cytogenetics (−5, −7, and complex abnormalities); the majority of the AML and MDS patients had intermediate cytogenetics (diploid and other cytogenetics). Most of the MDS patients (70%) had refractory anemia with excess blasts in transformation (RAEB-T) (Table 1) and can be classified as acute leukemia based on the WHO classification. However, since these patients are biologically different from those with AML based on the data presented here (sec below), we elected to keep them separate from the AML patients and not include them in any of the survival or response analysis. Due to the low numbers of patients with MDS, no survival analysis on this group of patients was performed.

TABLE 1
Characteristics of Patients with Acute Myeloid Leukemia (AML),
Myelodysplastic Syndrome (MDS), or Acute Lymphoblastic Leukemia (ALL).

| Characteristic | AML, n = 147 | | MDS, n = 27 | | ALL, n = 34 | |
|---|---|---|---|---|---|---|
| Median age, years (range) | 64 | (17-84) | 63 | (24-75) | 37 | (18-78) |
| Performance Status | | | | | | |
| 0-1 | 110 | | 1 | | 28 | |
| 2-4 | 35 | | 26 | | 6 | |
| Cytogenetics, | | | | | | |
| Favorable | 8 | | 0 | | | |
| Unfavorable | 51 | | 11 | | | |
| Intermediate | 88 | | 16 | | | |
| Hyperdiploid | | | | | 2 | |
| Hypodiploid | | | | | 2 | |
| Ph+ | | | | | 3 | |
| Other | | | | | 27 | |
| Median white blood cell count (range) × 10⁹/L | 3.8 | (0.4-161.0) | 2.6 | (0.8-23.9) | 7.8 | (0.9-74) |
| Median Hemoglobin, g/dL (range) | 7.8 | (3.4-13.1) | 7.4 | (2.2-11.0) | 8 | (4.0-15) |
| Median Platelets × 10⁹/L (range) | 54 | (6-377) | 36 | (10-270) | 84 | (11-485) |
| LDH (U/L) | 772 | (289-11064) | 474 | (254-2322) | 1020 | (352-15113) |
| FAB classification | | | | | | |

| | | | |
|---|---|---|---|
| M0-2 | 78 | | |
| M3 | 2 | | |
| M4-5 | 38 | | |
| M6/M7 | 5 | | |
| RARS | | 1 | |
| RAEB | | 5 | |
| RAEB-T | | 19 | |
| CMML | | 1 | |
| L1-L2 | | | 30 |
| L3 | | | 7 |
| Ph+ | | | 3 |

Abbreviations:
RARS, refractory anemia with ring sideroblasts;
RAEB, refractory anemia with excess blasts;
RAEB-T, refractory anemia with excess blasts in transformation;
CMML, chronic myelomonocytic leukemia.

The ALL patients were adult with median age of 37 and included 7 patients classified as having Burkitt-type ALL. Only 3 of these patients were positive for Philadelphia chromosome. These patients were treated with Hyper-CVAD (cyclophosphamide, vincristine, adriamycin, and dexamethasone).

Frozen plasma samples from these patients as well as from healthy controls were analyzed for the levels of ubiquitin and proteasome using chemiluminescent immunoassays. For the quantitation of ubiquitin, we used anti-polyubiquitin antibodies; therefore, only polyubiquitin was measured in our assay. Both assays were sensitive (100 pg/mL for proteasome and 2 ng/mL for ubiquitin), highly accurate (<15% recovery for both), and highly reproducible (<15% CV, inter-assay).

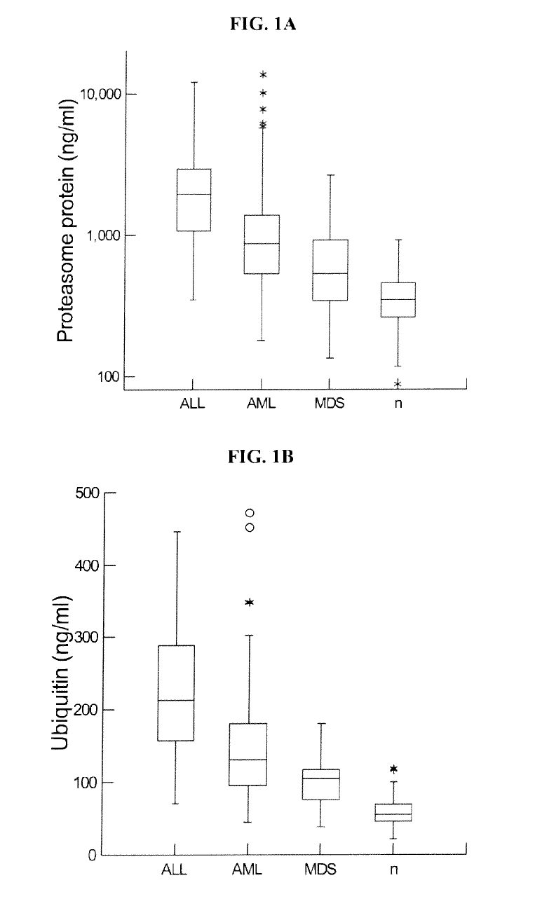

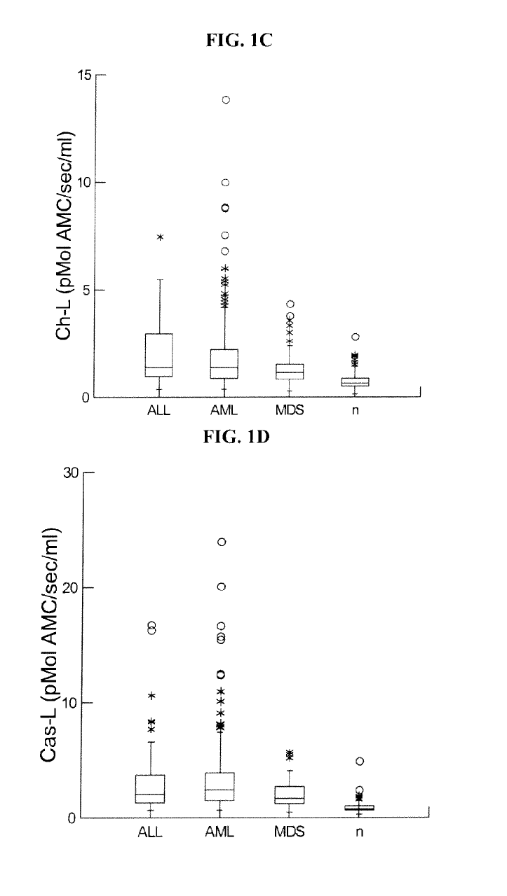

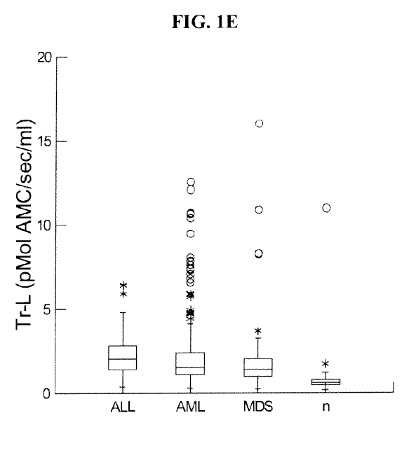

As shown in Table 2 and FIG. 1A, patients with leukemia or MDS had significantly higher levels of proteosome as compared with healthy controls (P<0.0001). Patients with ALL had the highest levels, significantly higher than in AML and MDS. A similar pattern was observed with ubiquitin (FIG. 1B) with highest levels in patients with ALL and lowest level in MDS. These patterns were somewhat different from the results of proteasome enzyme activities. While activities of Ch-L, Tr-L, and Cas-L were all elevated in AML, MDS, and ALL (P<0.0001) as compared with healthy controls (FIGS. 1C, 1D, 1E), Ch-L activity was not significantly different between AML, MDS, and ALL. Tr-L activity was significantly higher in ALL as compared with AML and MDS. In contrast, Cas-L activity was not different between AML and ALL, but significantly higher in AML and ALL than in MDS (FIG. 1D). This suggests that the UPS system plays a different role in each disease.

TABLE 2
Levels of proteasome, ubiquitin, and proteasome activities in
healthy controls, and patients with AML, ALL, or MDS.

| | Control (n = 96) | | | ALL (n = 34) | | | AML (n = 147) | | | MDS (n = 27) | | |
|---|---|---|---|---|---|---|---|---|---|---|---|---|
| | Median | Minimum | Maximum | Median | Minimum | Maximum | Median | Minimum | Maximum | Median | Minimum | Maximum |
| Proteasome | 336 | 87 | 813 | 2024 | 524 | 24786 | 875 | 179 | 13609 | 517 | 134 | 2646.00 |
| Ubiquitin | 53 | 21 | 118 | 253 | 98 | 446. | 132 | 45 | 471 | 99 | 39 | 179.80 |
| Ch-L/p | 2.13 | 0.16 | 12.18 | 0.74 | 0.04 | 5.28 | 1.67 | 0.37 | 7.01 | 2.13 | 0.83 | 8.63 |
| Tr-L/p | 1.93 | 0.41 | 18.18 | 0.96 | 0.05 | 5.48 | 1.58 | 0.13 | 33.10 | 2.50 | 0.22 | 62.05 |
| Cas-L/p | 2.61 | 0.50 | 12.77 | 0.88 | 0.10 | 3.61 | 2.94 | 0.79 | 14.00 | 3.55 | 1.38 | 8.06 |
| CH-L | 0.67 | 0.13 | 2.81 | 1.50 | 0.43 | 5.45 | 1.27 | 0.35 | 13.82 | 1.04 | 0.42 | 4.32 |
| Tr-L | 0.61 | 0.20 | 10.99 | 2.41 | 0.54 | 6.40 | 1.43 | 0.30 | 12.54 | 1.31 | 0.57 | 16.04 |
| Cas-L | 0.78 | 0.31 | 4.83 | 2.24 | 0.80 | 16.69 | 2.46 | 0.63 | 23.94 | 1.55 | 0.71 | 5.62 |

TABLE 3
Spearman correlations between proteasome and ubiquitin with various clinical and laboratory data.

| AML | | | | | | MDS | | | | | ALL | | | | |
|---|---|---|---|---|---|---|---|---|---|---|---|---|---|---|---|
| | Prtsm | Ubq. | Ch-L/P | Tr-L/P | Cas-L/P | Prtsm | Ubq. | Ch-L/P | Tr-L/P | Cas-L/P | Prtsm | Ubq. | Ch-L/P | Tr-L/P | Cas-L/P |
| Age | −0.03 | −0.07 | −0.04 | 0.02 | −0.03 | −0.11 | −0.16 | 0.00 | 0.02 | 0.07 | −0.08 | −0.27 | 0.19 | −0.09 | 0.17 |
| B2M | 0.31 | 0.23 | −0.13 | −0.28 | −0.03 | 0.32 | −0.07 | −0.06 | −0.23 | −0.10 | 0.54 | 0.37 | −0.29 | −0.45 | −0.31 |
| WBC | 0.36 | 0.46 | −0.18 | −0.22 | −0.02 | 0.40 | 0.15 | −0.32 | −0.35 | −0.29 | 0.46 | 0.41 | −0.61 | −0.21 | −0.55 |
| Bld | 0.30 | 0.25 | −0.16 | −0.19 | 0.01 | 0.38 | −0.07 | −0.39 | −0.41 | −0.35 | 0.62 | 0.48 | −0.57 | −0.47 | −0.68 |
| Blasts | | | | | | | | | | | | | | | |
| Platelets | 0.05 | 0.07 | −0.16 | 0.10 | −0.09 | −0.02 | 0.09 | 0.01 | −0.14 | −0.14 | −0.23 | −0.28 | −0.02 | −0.29 | 0.24 |
| HGB | −0.06 | 0.08 | −0.05 | 0.07 | −0.10 | −0.32 | −0.22 | −0.01 | 0.15 | 0.01 | −0.22 | −0.19 | 0.00 | 0.35 | 0.08 |
| Mrrw | 0.07 | 0.26 | 0.01 | −0.06 | 0.05 | 0.51 | 0.20 | −0.12 | −0.43 | −0.22 | −0.11 | 0.03 | −0.09 | 0.00 | 0.19 |
| Blasts | | | | | | | | | | | | | | | |
| BUN | 0.06 | 0.09 | 0.03 | −0.14 | 0.05 | −0.09 | −0.22 | −0.05 | −0.04 | −0.05 | −0.11 | −0.03 | 0.09 | −0.05 | −0.07 |
| Creati- | 0.10 | 0.06 | −0.12 | −0.21 | 0.01 | −0.08 | 0.00 | −0.10 | −0.17 | −0.20 | −0.10 | −0.07 | 0.15 | −0.04 | −0.10 |
| nine | | | | | | | | | | | | | | | |
| LDH | 0.56 | 0.32 | −0.17 | −0.45 | −0.04 | 0.31 | 0.11 | −0.01 | −0.13 | −0.15 | 0.74 | 0.69 | −0.52 | −0.58 | −0.57 |
| Ch-L | 0.80 | 0.37 | 0.05 | −0.55 | 0.06 | 0.76 | 0.64 | 0.12 | −0.54 | −0.13 | 0.47 | 0.34 | 0.12 | −0.38 | −0.02 |
| Tr-L | 0.20 | 0.15 | −0.07 | 0.47 | −0.05 | −0.20 | 0.06 | 0.36 | 0.71 | 0.46 | 0.01 | 0.15 | 0.02 | 0.54 | −0.05 |
| Cas-L | 0.80 | 0.45 | −0.07 | −0.56 | 0.19 | 0.73 | 0.54 | 0.06 | −0.39 | −0.03 | 0.44 | 0.25 | −0.07 | −0.44 | 0.22 |
| Protsm | | 0.42 | −0.49 | −0.72 | −0.37 | | 0.43 | −0.48 | −0.77 | −0.64 | | 0.77 | −0.75 | −0.80 | −0.74 |
| Ubiq- | 0.42 | | −0.16 | −0.29 | 0.00 | 0.43 | | 0.23 | −0.20 | −0.07 | 0.77 | | −0.50 | −0.47 | −0.65 |
| uitin | | | | | | | | | | | | | | | |
| Ch-L/P | −0.49 | −0.16 | | 0.39 | 0.75 | −0.48 | 0.23 | | 0.56 | 0.83 | −0.75 | −0.50 | | 0.61 | 0.75 |
| Tr-L/P | −0.72 | −0.29 | 0.39 | | 0.32 | −0.77 | −0.20 | 0.56 | | 0.72 | −0.80 | −0.47 | 0.61 | | 0.52 |
| Cas-L/P | −0.37 | 0.00 | 0.75 | 0.32 | | −0.64 | −0.07 | 0.83 | 0.72 | | −0.74 | −0.65 | 0.75 | 0.52 | |

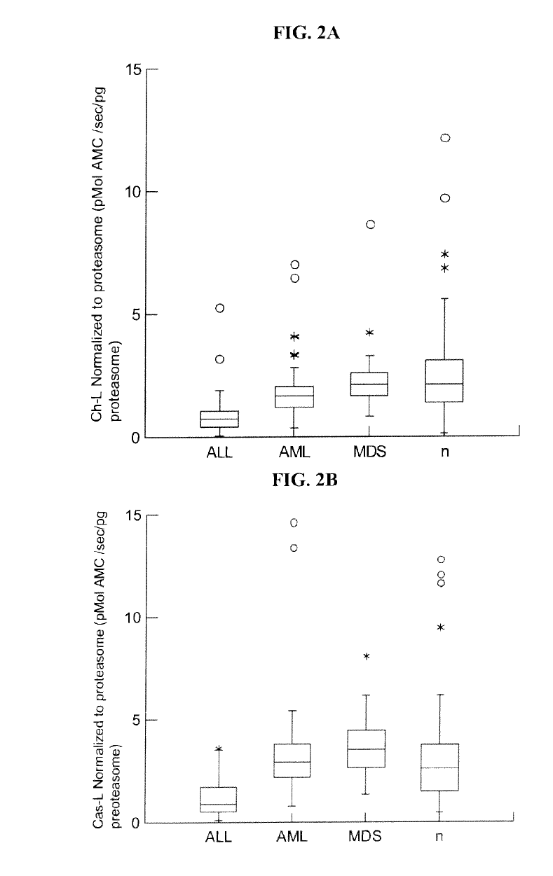

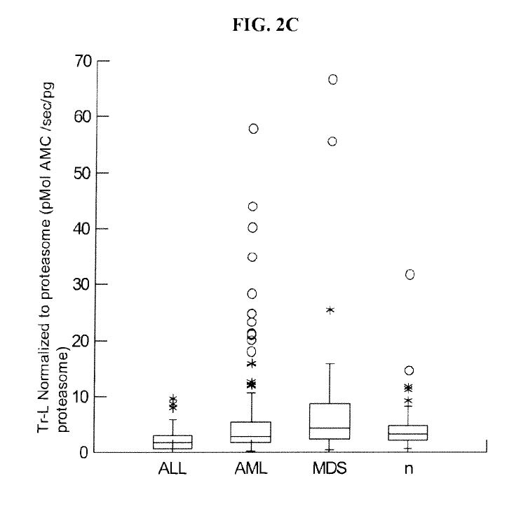

To explore the differences in UPS profiles between different hematopoietic disorders, the level of enzymatic of activity for each proteasomal enzyme was normalized to the level of proteasome protein by dividing the proteasome enzymatic activities by the proteasome concentration. These levels were then compared between AML, ALL, and MDS and the healthy control group (FIG. 2). The circulating proteasomes had Ch-L specific activity (Ch-L/p) that was significantly lower in ALL and AML than in healthy controls, whereas the proteasomes in MDS patients had similar specific activity to those in healthy controls (FIG. 2A). Furthermore, proteasomes in patients with ALL had significantly lower levels of Ch-L/p than in patients with AML (FIG. 2A). In contrast, Cas-L specific activity (Cas-L/P) was similar in AML and healthy controls, but was significantly higher in patients with MDS than controls and AML (FIG. 2B). Patients with ALL had significantly lower levels of Cas-L/p than healthy controls or AML (FIG. 2B). The circulating proteasomes had Tr-L specific activity that varied between diseases (FIG. 2C). ALL had the lowest levels of Tr-L/p, significantly lower than AML and healthy controls. Patients with MDS had significantly higher levels of Tr-L/p than healthy controls and AML. There was no significant difference in Tr-L/p levels between AML patients and healthy controls (FIG. 2C). These data suggest that the specific activities of each proteasome are in general lower in leukemia than in normal cells, but more proteasomes are present in leukemic cells leading to more net enzymatic activity in leukemic cells.

### Predicting Clinical Behavior

We explored the ability of proteasome protein levels or specific activity to predict response to therapy, relapse, and survival. Proteasome level as a continuous variable predicted response to therapy in AML (P=0.04), but not in ALL (data not shown). Ubiquitin levels did not predict response in AML nor ALL. Using the median proteosome level (875 ng/mL) as a cut-off, AML patients with proteasome levels less than median had significantly better response rate than those with higher levels (P=0.01). As for survival, Table 4 lists the statistically significant predictors of survival in AML and ALL. Patients with MDS were too few for survival analysis.

TABLE 4
Cox regression model for predicting survival and complete remission duration
(CRD) in Patients with Acute Myeloid Leukemia and Acute lymphoblastic leukemia.

| | | Standard | | Wald | |
|---|---|---|---|---|---|
| | Beta | Error | t-value | Statistic | P-value |
| Univariate Survival as predicted by | | | | | |
| proteasome protein in AML | | | | | |
| In all patients | 0.0003 | 0.0001 | 4.4383 | 19.6988 | <0.00001 |
| In Intermediate cytogenetic group | 0.000190 | 0.000086 | 2.222028 | 4.937406 | 0.020 |
| In poor Cytogentic group | 0.000298 | 0.000087 | 3.418611 | 11.68690 | 0.0006 |
| Multivariate in AML patients | | | | | |
| Proteasome | 0.0002 | 0.0001 | 3.0434 | 9.2622 | 0.002 |
| Cytogenetic grouping | 0.7860 | 0.1859 | 4.2286 | 17.8812 | <0.00001 |
| Age grouping (<70 vs >70) | 0.6489 | 0.1986 | 3.2673 | 10.6751 | 0.001 |
| Performance status (<2 vs >2) | 0.6099 | 0.2130 | 2.8631 | 8.1974 | 0.004 |
| CRD in AML | | | | | |
| Ch-L/P | 0.2956 | 0.1678 | 1.7619 | 3.1041 | 0.08 |
| Cas-L/P | 0.1460 | 0.0821 | 1.7774 | 3.1591 | 0.08 |
| Univariate, survival in ALL | | | | | |
| Proteasome | 0.00001 | 0.0000 | 1.6713 | 2.7934 | 0.09 |
| Ch-L/P | −1.3914 | 0.6323 | −2.2005 | 4.8422 | 0.03 |
| Cas-L/P | −0.7028 | 0.3980 | −1.7661 | 3.1191 | 0.08 |
| Univariate, CRD in ALL | | | | | |
| Ubiquitin | 0.0051 | 0.0027 | 1.9299 | 3.7247 | 0.05 |
| Ch-L/P | −1.1975 | 0.6623 | −1.8083 | 3.2699 | 0.07 |
| Cas-L/P | −1.8295 | 0.7336 | −2.4939 | 6.2195 | 0.01 |

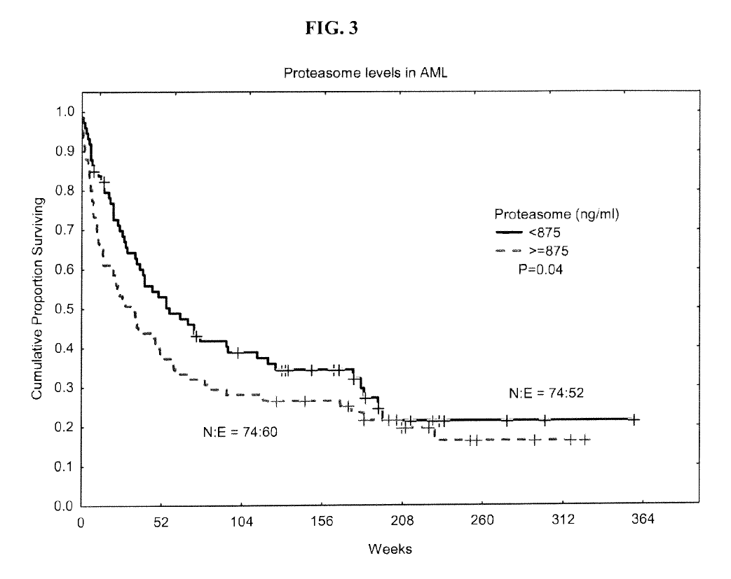

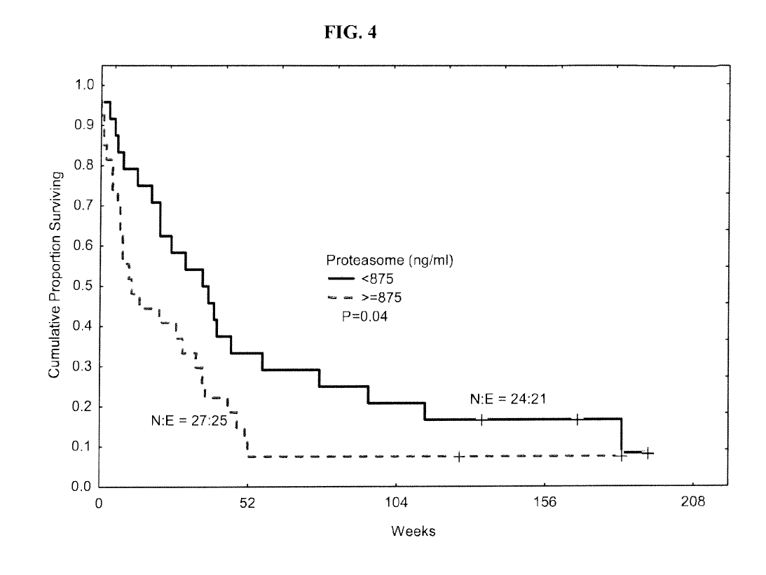

As shown in Table 4, using Cox proportional hazard model, proteasome, but not ubiquitin levels, were strong predictors of survival when used as continuous variable (P<0.0001) (Table 4). Even when we considered AML patients in the unfavorable cytogenetic group (n=51), proteasome levels were strong predictors of survival in this group of patients (P=0.0006). The proteasome level was also predictive of survival in the intermediate cytogenetic group (P=0.03) (Table 4). In addition to proteosome levels, multivariate analysis was performed incorporating the major factors that are known to be predictors of survival in AML including cytogenetic grouping, age grouping, and performance status. Proteasome levels remained a strong predictor of survival independent of all other factors (P=0.002) (Table 4). Furthermore, when the median was used as a cut-off, patients with levels higher than the median had significantly shorter survival (P=0.04) (FIG. 3). Restricting the analysis to only patients with unfavorable cytogenetics also showed that patients with proteasome levels >875 ng/mL had significantly shorter survival (P=0.04) (FIG. 4).

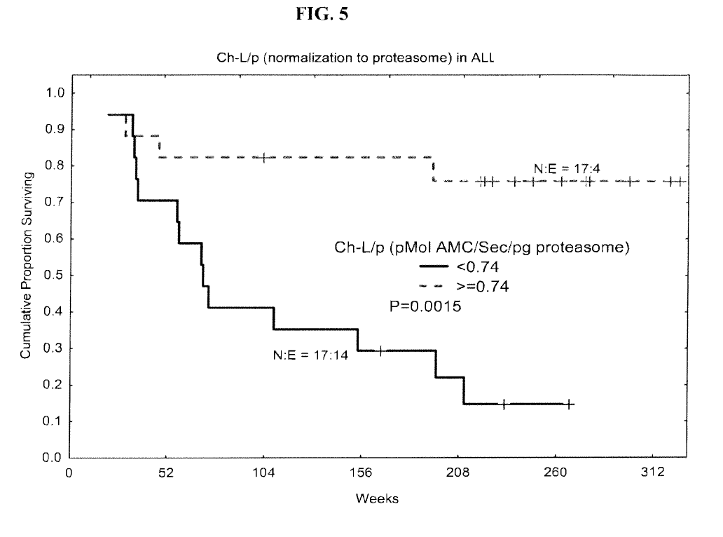

Ch-L/p and Cas-L/p showed a marginal correlation with survival in AML (P=0.08 for both) (Table 4). There was a significant negative correlation between Ch-L/p as a continuous variable and survival (P=0.03) (Table 4). The Cas-L/p specific activity showed a slight, but not significant, negative correlation with survival in ALL (P=0.08). When we used the median as cut-off point, ALL patients with Ch-L/p level <0.74 (pMol AMC/sec/pg proteasome) had significant longer survival (P=0.0015) (FIG. 5). Using the median for Cas-L/p, we also found a negative correlation with survival (P=0.03) in ALL. Patients with lower than the median Cas-L/p (0.88 pMol AMC/sec/pg proteasome) had significantly longer survival.

Complete remission duration (CRD) in AML did not correlate with any of the proteasome or ubiquitin parameters, but in ALL, ubiquitin levels as continuous variable correlated significantly with CRD (P=0.05) (Table 4). Unlike with survival, Cas-L/p correlated negatively better than Ch-L/p with CRD (P=0.01 vs. P=0.07) as continuous variables (Table 4).

### Example 2

### Ubiquitin as a Prognostic Indicator in Patients with CLL

The ubiquitin-proteasome pathway is implicated in the pathogenesis of many hematological malignancies. We measured ubiquitin protein level in the plasma of CLL patients and correlated levels with clinical behavior. Using the Meso Scale Discovery (MSD) platform (described in Example 1), we quantified poly-ubiquitin levels in plasma samples from 138 patients with CLL and compared levels with 101 normal control patients. The results were compared with various laboratory parameters and outcomes.

Patients with CLL had significantly (P<0.001) higher levels of poly-ubiquitin proteins (median: 158.2, range 35.0-281.2) as compared normal control (median: 57.3, range 22.0-160.2). Poly-ubiquitin levels showed no correlation with Rai stage, performance status, beta-2 microglobulin (B2M), or the mutation status of the IgVH. However, the poly-ubiquitin levels correlated negatively with survival when considered as a continuous variable (P=0.04).

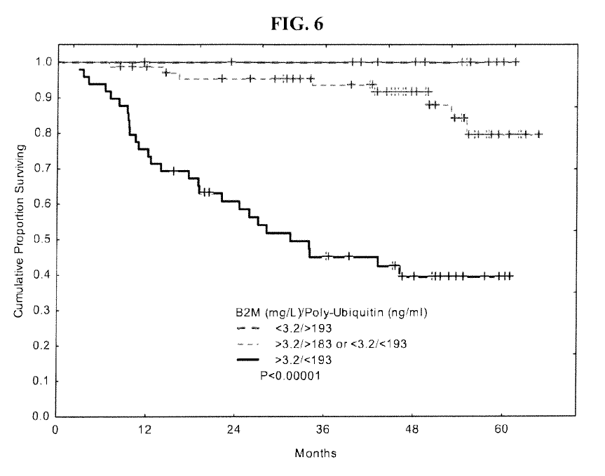

In a multivariate model incorporating IgVH mutation status, B2M and poly-ubiquitin, only B2M (P=0.00004) and poly-ubiquitin P=0.04), but not IgVH mutation status (P=0.12), were independent predictors of survival. Using a cut-off point corresponding to the upper quartile also showed that patients with ubiquitin levels higher than 192 ng/ml have significantly better survival (FIG. 6).

Using both B2M and poly-ubiquitin allows us to stratify patients to three different groups: (1) the favorable outcome group with high poly-ubiquitin and low B2M, (2) the unfavorable outcome group with low poly-ubiquitin and high B2M, and (3) the intermediate outcome group (P<0.00001). These data demonstrate the importance of the proteasome-ubiquitin system in the pathophysiology of CLL. The data further show that the poly-ubiquitin levels in the plasma of patients can he used as a prognostic factor.

The contents of the articles, patents, and patent applications, and all other documents and electronically available information mentioned or cited herein, are hereby incorporated by reference in their entirety to the same extent as if each individual publication was specifically and individually indicated to he incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Thus, for example, the terms “comprising”, “including.” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

### Example 3

### UPS Profiling in Patients with AML, ALL, CLL, CML and MDS

To further explore the use of the UPS signature model in the diagnosis of various hematological disorders, the UPS profile in the plasma of patients with acute myeloid leukemia (AML) (n=111), acute lymphoblastic leukemia (ALL) (n=29), advanced myelodysplastic syndrome (MDS) (n=20), chronic lymphocytic leukemia (CLL) (n=118), chronic myeloid leukemia (CML) (n=128), and plasma from 85 healthy normal control subjects was analyzed. The CML group included 46 patients in accelerated/blast crisis (ACC/BL) and 82 cases in the chronic phase (Ch).

To profile the UPS system, we measured the proteasome and ubiquitin (poly-ubiquitin) and the three enzymatic activities of the proteasome (Ch-L, Cas-L, and Tr-L), in addition, we normalized these three enzymatic activities to the levels of proteasome protein in the plasma to measure the specific activity of these enzyme generating the following three parameters: Ch-L/p, Cas-L/p, and Tr-L, respectively (See Example 1), This generated 8 variables that were used in multivariate logistic regression models for the differential diagnosis between various leukemic processes.

The UPS signature was easily able to differentiate between patients with a leukemic process and normal controls using 6 different variables (Tr-L/P, Ch-L, Ch-L/p, Cas-L, Cas-L/P, and ubiquitin) with AUC of 0.9912. Distinguishing between acute process (AML, ALL, and MDS) vs. chronic (CML and CLL) was less efficient with AUC of 0.8533 using the following variables variable (Tr-L, Tr-L/P, Cas-L/P, Ch-L/P, Proteasome, Ch-L). Most likely this is due to the presence of significant number (36%) of patients in the ACC/BL phase of CML. Collectively, these data show that the UPS was very powerful in distinguishing between individual leukemias (Table 5). This data does not only support the concept that the UPS profile is unique for each leukemic process, but also suggests that this profile can be used as biomarker for the differential diagnosis of between leukemias.

TABLE 5
UPS Signature Model in the Diagnosis of IIematological Disorders

| Comparison | #Var | Analytes | N | #Case | #CTRL | AUC |
|---|---|---|---|---|---|---|
| MDS vs Norml | 6 | Proteasome, Ubiq, Cas-L, Tr-L, | 105 | 20 | 85 | 0.98 |
| | | Cas−L/P Ch-LP | | | | |
| MDS vs ALL | 4 | Proteasome, Ubiq, Tr-L, Cas- | 49 | 20 | 29 | 0.9845 |
| | | L/P | | | | |
| MDS vs AML | 7 | Proteasome, Ubiq, Ch-L, Tr- | 131 | 20 | 111 | 0.791 |
| | | L, Cas-LP, Ch-L/P, Tr-L/P | | | | |
| MDS vs CLL | 2 | Cas-L/P, Ch-L/P | 163 | 27 | 136 | 0.9909 |
| MDS vs CML | 3 | Ubiq, Cas-L/P, Tr-L | 147 | 20 | 127 | 0.9496 |
| MDS vs CML (Chronic) | 4 | Ubiq, Cas-L/P, Tr-L, Tr-L/P | 101 | 20 | 81 | 0.9608 |
| MDS vs CML | 5 | Ubiq, Cas-LP, Tr-L, Tr-LP, | 66 | 20 | 46 | 0.9516 |
| (ACC/BL) | | Ch-L | | | | |
| AML vs Norm | 5 | Ubiq, Cas-L, Tr-L, Tr-L/P, | 196 | 111 | 85 | 0.9884 |
| | | Ch-L/P | | | | |
| AML vs ALL | 5 | Cas-L/P, Ch-L/P, Ubiq, Ch- | 140 | 111 | 29 | 11.9503 |
| | | L/P, Proteasome | | | | |
| AML vs CLL | 2 | Cas-L/P, Ch-L/P | 283 | 147 | 136 | 0.9835 |
| | | Tr-L/P, Tr-L, Ubiq, Cas-L/P, | | | | |
| AML vs CML | 6 | Cas-L, Ch-LP | 238 | 111 | 127 | 0.8301 |
| AML vs CML | 6 | Tr-L/P, Tr-L, Ubiq, Cas-LP, | 192 | 111 | 81 | 0.8396 |
| (Chronic) | | Ch-LP, Proteasome | | | | |
| AML vs CML | 6 | Tr-L/P, Tr-L, Ubiq, Cas-LP, | 157 | 111 | 46 | 0.8239 |
| (ACC/BL) | | Ch-LP, Cas-L | | | | |
| ALL, vs Norm | 2 | Ubiq, Tr-L | 114 | 29 | 85 | 0.9984 |
| | | Ubiq, Tr-L, Tr-L/P, Ch-L, | | | | |
| ALL vs CLL | 6 | Ch-L/P, Cas-LP | 147 | 29 | 118 | 0.9031 |
| ALL vs CML | 7 | no Tr-LP | 156 | 29 | 127 | 0.9005 |
| ALL vs CML (Chronic) | 7 | no Tr-L | 110 | 29 | 81 | 0.9264 |
| ALL vs CML | 7 | no Tr-L | 75 | 29 | 46 | 0.8909 |
| (ACC/BL) | | | | | | |
| CLL vs Norm | 3 | Cas-L/P, Ch-L, Ubiq | 203 | 118 | 85 | 0.9978 |
| | | Cas-L/P, Cas-L, Ch-L/P, Tr- | | | | |
| CLL vs CML | 5 | L, Ubiq | 245 | 118 | 127 | 0.9746 |
| | | Cas_LP Cas_L Ch_LP Tr_L | | | | |
| CLL vs CML (Chronic) | 5 | Ubiq | 199 | 118 | 81 | 0.9749 |
| CLL vs CML | 5 | Cas-L/P, Cas-L, Ch-L/P, Tr- | 164 | 118 | 46 | 0.9746 |
| (ACC/BL) | | L, Ubiq | | | | |
| CML vs Norm | 2 | Ubiq, Tr-L | 212 | 127 | 85 | 0.9986 |

What is claimed is:

1. A method for diagnosing a proliferative hematological disorder in a subject, the method comprising: determining, in an acellular body fluid sample from the subject, the specific activity of one or more proteasomal peptidases selected from the group consisting of chymotrypsin-like activity (Ch-L), trypsin-like activity (Tr-L), and caspase-like activity (Cas-L), wherein the specific activity is determined by normalizing the one or more peptidase activities to the amount of proteasomal protein in the sample, and diagnosing the subject as having a proliferative hematological disorder when a difference of the specific activity of one or more proteasomal peptidases compared to a reference specific activity indicates a proliferative hematological disorder in the subject.

2. The method of claim 1, wherein the proliferative hematological disorder is selected from the group consisting of chronic lymphocytic leukemia (CLL), acute myeloid leukemia (AML), myelodysplastic syndrome (MDS), and acute lymphoblastic leukemia (ALL).

3. The method of claim 1, wherein the acellular body fluid is selected from the group consisting of serum and plasma.

4. The method of claim 1, wherein the reference specific activity is a cutoff value determined from the specific activity of one or more proteasomal peptidases present in a comparable sample from healthy individuals, and wherein an increase or decrease in the subject specific activity relative to the cutoff value is used to determine a diagnosis for the subject.

5. The method of claim 1, wherein a determined specific activity of Ch-L (Ch-L/p) in the subject sample that is lower than a reference specific activity indicates a diagnosis of AML or ALL for the subject.

6. The method of claim 1, wherein a determined specific activity of Cas-L (Cas-L/p) in the subject sample that is higher than a reference specific activity indicates a diagnosis of MDS for the subject.

7. The method of claim 1, wherein a determined specific activity of Cas-L (Cas-L/p) in the subject sample that is lower than a reference specific activity indicates a diagnosis of ALL for the subject.

8. The method of claim 1, wherein a determined specific activity of Tr-L (Tr-L/p) in the subject sample that is lower than a reference specific activity indicates a diagnosis of ALL for the subject.

9. The method of claim 1, wherein a determined level of specific activity of Tr-L (Tr-L/p) in the subject sample that is higher than a reference specific activity indicates a diagnosis of MDS for the subject.

10. The method of claim 1, wherein said method further comprises measuring the amount of ubiquitin in the subject sample.

11. A method of diagnosing a proliferative hematological disorder in a subject, the method comprising:
determining the amount of proteasomal protein in a test sample for the subject;
determining the amount of one or more proteasomal peptidase activities in a test sample from the subject, the peptidase activities selected from the group consisting of chymotrypsin-like activity (Ch-L), trypsin-like activity (Tr-L), and caspase-like activity (Cas-L),
normalizing the amount of one or more proteasomal peptidase activities to the amount of proteasomal protein to provide a specific activity of the one or more proteasomal peptidases; and
diagnosing the subject as having a proliferative hematological disorder when the specific activity of at least one or more proteasomal peptidases is different from a reference value for the specific activity of that proteasomal peptidase in disease-free subjects.

12. The method of claim 11, wherein the test sample is an acellular body fluid sample.

13. The method of claim 12, wherein the acellular body fluid is selected from the group consisting of serum and plasma.

14. The method of claim 11, wherein the test sample is a cell-containing sample.

15. The method of claim 11, wherein the reference value is a cutoff value determined from the specific activity of one or more proteasomal peptidases present in a comparable sample from disease-free individuals, and wherein an increase or decrease in the subject specific activity relative to the cutoff value is used to determine a prognosis for the subject.

16. The method of claim 11, wherein the proliferative hematological disorder is selected from the group consisting of chronic lymphocytic leukemia (CLL), acute myeloid leukemia (AML) myelodysplastic syndrome (MDS), and acute lymphoblastic leukemia (ALL).

17. The method of claim 11, wherein the specific activity of Ch-L (Ch-L/p) in the subject sample that is lower than a reference value indicates a diagnosis of AML or ALL for the subject.

18. The method of claim 11, wherein the specific activity of specific activity of Cas-L (Cas-L/p) in the subject sample that is higher than a reference value indicates a diagnosis of MDS for the subject.

19. The method of claim 11, wherein the specific activity of specific activity of Cas-L (Cas-L/p) in the subject sample that is lower than a reference value indicates a diagnosis of ALL for the subject.

20. The method of claim 11, wherein the specific activity of specific activity of Tr-L (Tr-L/p) in the subject sample that is lower than a reference value indicates a diagnosis of ALL for the subject.

21. The method of claim 11, wherein the specific activity of specific activity of Tr-L (Tr-L/p) in the subject sample that is higher than a reference value indicates a diagnosis of MDS for the subject.

22. The method of claim 11, wherein said method further comprises measuring the amount of ubiquitin in the subject sample,

23. A method of determining a prognosis of a subject having a proliferative hematological disorder, wherein the method comprises:
determining the specific activity of one or more proteasomal peptidases selected from the group consisting of chymotrypsin-like activity (Ch-L), trypsin-like activity (Tr-L), and caspase-like activity (Cas-L), wherein the specific activity is determined by normalizing the one or more proteasomal peptidases activities to the amount of one or more proteasomal proteins in the sample, and providing a prognosis for the subject based on a difference of the specific activity of one or more peptidases compared to a reference level.

24. The method of claim 23, wherein the reference level is the specific activity of the corresponding one or more proteasomal proteins in a comparable sample from one or more healthy individuals.

25. The method of claim 23, wherein the test sample is a cell-containing sample.

26. The method of claim 23, wherein the test sample is an acellular body fluid sample.

27. The method of claim 26, wherein the body fluid is selected from the group consisting of serum and plasma.

28. The method of claim 23, wherein the prognosis is selected from the group consisting of survival rate, 5-year survival rate, and complete remission duration (CRD).

29. The method of claim 23, wherein the disease or disorder is a proliferative hematological disorder.

30. The method of claim 29, wherein the proliferative hematological disorder is selected from the group consisting of AML, CLL, ALL and MDS and the prognosis is survival rate.

31. The method of claim 23, wherein the specific activity of Ch-L (Ch-L/p) in the subject sample that is higher than a reference value indicates a better survival rate from the disorder.

32. The method of claim 23, wherein the specific activity of Ch-L (Ch-L/p) or Cas-L (Cas-L/p) in the subject sample that is higher than a reference value indicates a better survival rate from the disorder.

33. The method of claim 23, wherein said method further comprises measuring the amount of ubiquitin in the subject sample.

34. A method of determining a prognosis of a subject having a proliferative hematological disorder, wherein the method comprises:
determining the level of circulating ubiquitin or polyubiquitin in a sample from the subject, and providing a prognosis for the subject based on a difference of the level of circulating ubiquitin or polyubiquitin compared to a reference level.

35. The method of claim 34, wherein the proliferative hematological disorder is CLL.

36. The method of claim 35, wherein a level of circulating ubiquitin or polyubiquitin greater than about 192 ng/mL indicates a better survival rate for the subject compared to subjects having a level of circulating ubiquitin or polyubiquitin less than about 192 ng/mL.

37. The method of claim 34 further comprising determining the level of beta-2 microglobulin in a sample from the subject.

38. The method of claim 37, wherein a level of beta-2 microglobulin less than about 3.2 mg/L indicates a better survival rate for the subject compared to subjects having a level of circulating beta-2 microglobulin greater than about 3.2 mg/L.
